(19) United States
(12) Patent Application Publication
(10) Pub. No.: US 2020/0399308 A1
(43) Pub. Date: Dec. 24, 2020

(54) COMPOUNDS AND METHODS FOR ELICITING ANTIMICROBIAL ACTIVITY

(71) Applicant: Technion Research & Development Foundation Limited, Haifa (IL)

(72) Inventor: Amram MOR, Nesher (IL)

(73) Assignee: Technion Research & Development Foundation Limited, Haifa (IL)

(21) Appl. No.: 16/975,765

(22) PCT Filed: Mar. 2, 2018
(86) PCT No.: PCT/IL2018/050237

§ 371 Date: Aug. 26, 2020

(51) Int. Cl. C07K 5/02; A61P 31/06; A61K 31/43

(52) U.S. Cl. CPC C07K 5/02; A61K 31/43; A61P 31/06

(57) ABSTRACT

Non-antimicrobial compounds, methods and compositions comprising the same for treating medical conditions associated with pathogenic microorganism in a subject, as well as drug-resistant strains thereof, which are effective in immunopotentiating the pathogenic microorganism to the antimicrobial systems in the subject, and/or act in synergism with exogenous antimicrobial drugs.

### FIELD AND BACKGROUND OF THE INVENTION

The present invention, in some embodiments thereof, relates to non-antibiotic pharmaceutically active compounds, compositions, uses and methods of treatments using the same, and more particularly, to compounds that elicit an improved host-mediated antimicrobial activity, and potentiate antimicrobial drugs against microorganisms including drug-resistant microorganisms.

Antibiotics, which are also referred to herein and in the art as antibacterial or antimicrobial agents, constitute one of the greatest triumphs of modern medical science, ever since their discovery and recognition by Alexander Fleming in 1928. Natural and synthetic antimicrobial agents have been developed and used for decades with great success and virtually transformed the survival rates of infected subjects all over the world. However, over the decades, almost all the prominent infection-causing bacterial strains (pathogenic microorganisms) have developed resistance, at least to some degree, to currently available antibiotics.

WO/2006/035431 and WO/2008/132737 teach a class of antimicrobial compounds, primarily composed of fatty acid and lysine residues that exhibit high antimicrobial activity, low resistance induction, non-hemolyticity, plasma proteases resistibility, and high affinity to microbial membranes.

WO/2008/132738 teach a class of compounds, primarily composed of fatty acid and lysine residues that exhibit activity against cancerous cells.

WO/2009/090648 disclose methods and compositions for treating microbial infections associated with an emergence of resistance of a pathogenic microorganism to an antimicrobial agent, following treatment with antimicrobial agent. The methods are effected by using a compound which exhibits antimicrobial re-sensitizing activity, for re-sensitizing the pathogenic microorganisms to the antimicrobial agent, in combination with the antimicrobial agent.

Other documents teaching aspects of these biologically active compounds, based on ω-amino-fatty acid and positively charged amino acid residues, include WO/2008/072242, teaching compositions and methods for concentrating and depleting microorganisms and WO/2011/016043, teaching compositions-of-matter comprising compound-mediated cochleates, which can co-encapsulate other bioactive agents as a delivery vehicle.

### SUMMARY OF THE INVENTION

The present invention, in some embodiments thereof, relates to non-antibiotic pharmaceutically active compounds, compositions, uses and methods of treatments using the same, and more particularly, to compounds that elicit an improved host-mediated antimicrobial activity, and potentiate antimicrobial drugs against microorganisms including drug-resistant microorganisms.

Provided herewith are compounds that inflicted outer membrane damage at a low micromolar range, whereas measurable bacterial growth inhibition in broth medium required more than 10-fold higher concentrations. In serum, however, the compounds induced antibacterial activity in a manner suppressible by anticomplement antibodies or heat treatment and acted synergistically with exogenous lysozyme in broth and serum media. Upon subcutaneous administration, the compounds provided herein exhibited high circulating levels that correlated with significant therapeutic efficacies, using either the mouse peritonitis-sepsis model or the thigh infection model. These findings are consistent with the view that, by damaging the outer membrane, these compounds were able to enhance pathogen's susceptibility, e.g., gram-negative bacilli, to antibacterial components of the immune humoral arm. Such compounds are useful in fighting pathogenic threats through sensitization of the microorganism to endogenous and/or exogenous antibacterial proteins such as lysozyme and complements, as well as to antimicrobial agents (antibiotic drugs), while exhibiting no antimicrobial activity per se, and low toxicity.

According to one aspect of some embodiments of the present invention, there is provided a compound selected from the group consisting of:

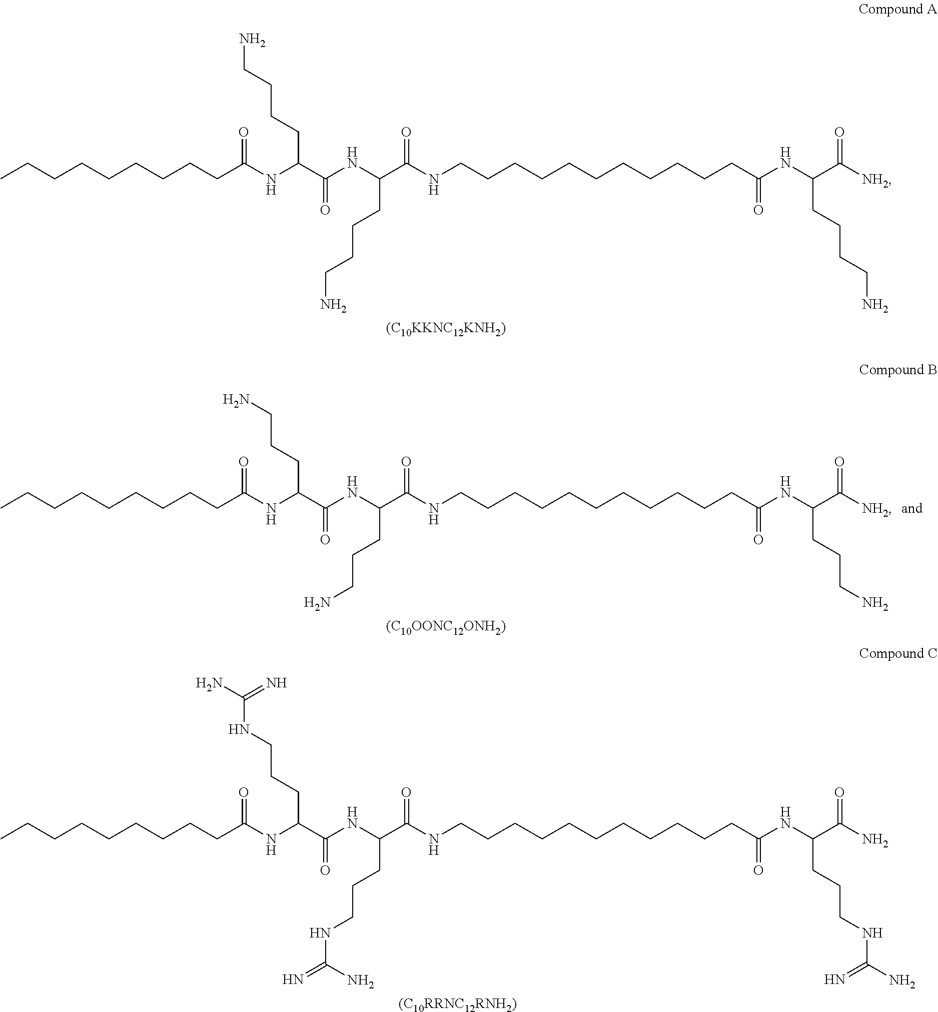

According to some embodiments of the invention, the compound is Compound A. According to some embodiments of the invention, the compound is Compound B. According to some embodiments of the invention, the compound is Compound C. The compounds described herein have unique features that enable to use these compounds as immunopotentiating agents, antimicrobial agent potentiating agents or microbial re-sensitization agents. The present aspects and embodiments thereof further encompass methods and compositions using any enantiomers, prodrugs, solvates, hydrates and/or pharmaceutically acceptable salts of the compounds described herein.

According to an aspect of some embodiments of the present invention, there is provided a pharmaceutical composition that includes, as an active ingredient, any one or more of the compounds presented herein, or any enantiomer, prodrug, solvate, hydrate and/or pharmaceutically acceptable salt thereof, and a pharmaceutically acceptable carrier.

According to some embodiments of the invention, the pharmaceutical composition is packaged in a packaging material and identified in print, in or on the packaging material, for use in the treatment of a medical condition associated with a pathogenic microorganism in a subject.

According to some embodiments, the pharmaceutical composition is essentially devoid of an antimicrobial agent.

According to some embodiments, the pharmaceutical composition further includes an antimicrobial agent.

According to some embodiments, the antimicrobial agent is ampicillin and the pathogenic microorganism is Yersinia pseudotuberculosis.

According to an aspect of some embodiments of the present invention, there is provided a method of treating a medical condition associated with a pathogenic microorganism in a subject, the method includes administering to the subject a therapeutically effective of any one or more of the compounds presented herein, or any enantiomer, prodrug, solvate, hydrate and/or pharmaceutically acceptable salt thereof.

According to some embodiments, the method is essentially devoid of administering an antimicrobial agent to the subject. In some embodiments, the therapeutically effective of the compound is an immunopotentiating amount.

According to some embodiments, the method further includes co-administering to the subject a therapeutically effective amount of an antimicrobial agent, and co-administering to the subject a therapeutically effective amount of the compound presented herein, wherein the therapeutically effective amount of the antimicrobial agent is lower than a therapeutically effective amount thereof when administered alone, without the compound, and the therapeutically effective amount of compound is a potentiating amount thereof with respect to the antimicrobial agent.

According to an aspect of some embodiments of the present invention, there is provided a use of the compound presented herein as an active ingredient in the preparation of a medicament for treating a medical condition associated with a pathogenic microorganism in a subject.

According to an aspect of some embodiments of the present invention, there is provided a use of the compound presented herein as an active ingredient in the preparation of a medicament for sensitizing a pathogenic microorganism to an antimicrobial agent.

In some embodiments, the medicament further include the antimicrobial agent.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C present the results of the membrane damage and bioavailability assessments, showing dose-dependent permeabilization of the outer (FIG. 1A) and cytoplasmic (FIG. 1B) membranes of the Escherichia coli mutant ML-35p, as determined in buffer, 16 minutes after addition of the compounds presented herein, wherein dermaseptin (25 μM) was used as positive control, representing full permeabilization, and the insets show representative kinetics at 12.5 μM, and further showing plasma concentrations (FIG. 1C) determined by liquid chromatography-mass spectrometry after subcutaneous administration (12.5 mg/kg body weight) to ICR mice (squares denote Compound A, triangles denote Compound B, circles denote Compound C, Xs denote vehicle control and diamonds denote dermaseptin; data are for 2 mice/time point; error bars represent standard deviations);

FIGS. 2A-B present results of antibacterial activity assays of mouse serum, wherein FIG. 2A shows bacterial survival in 80% serum inoculated with Escherichia coli 25922 (Ec) (0.9±0.2)×10³ CFU/mL or Klebsiella pneumoniae 1287 (Kp) (1.08±0.21) □10³ CFU/mL, treated with PBS vehicle (control) or 10 μM Compound B and incubated for 3 h (37 □) in absence or presence of anti-complements C5/C5a mouse antibody (AB), and FIG. 2B shows bacterial survival under roughly similar conditions (i.e., after 3 h incubation in 80% serum) when the serum was obtained 1 hour after subcutaneous administration of the tested compound as described in FIG. 1C, followed by E. coli 25922 inoculation and culture as in FIG. 2A (plot also shows a duplicated sample subjected to heat-treatment (HT); error bars represent standard deviations from the mean);

FIGS. 3A-D present evidence of synergism of Compound B and lysozyme (LZ) in broth and serum, wherein FIG. 3A and FIG. 3B show the results of the checkerboard assay for bacterial growth inhibition in broth medium containing a mean inoculum (±SD) of 1.1×10⁴±0.05×10⁴ colony-forming units (CFU)/mL of Escherichia coli 25922 (FIG. 3A) or 1.2×10⁴±0.08□10⁴ CFU/mL of Klebsiella pneumoniae 1287 (FIG. 3B), and wherein FIG. 3C and FIG. 3D show the survival of serum-resistant E. coli 25922 and K. pneumoniae 1287 in fresh mouse serum supplemented with 10 μM of Compound B, 18 μM of LZ, or 13 μM of lactoferrin (LF) alone, combination of Compound B and LZ, or combination of Compound B and LF (empty squares denote LZ, top-filled squares denote 2.5 μM Compound B plus LZ, left-filled squares denote 5 μM Compound B plus LZ, full squares denote 10 μM Compound B plus LZ, circles denote vehicle control, triangles denote 10 μM Compound B, empty diamonds denote LF and full diamonds denote 10 μM Compound B plus LF; error bars represent standard deviations from the mean);

FIGS. 4A-C show antibacterial properties of human serum, wherein FIG. 4A shows growth kinetics of serum-resistant Escherichia coli 25922 in normal serum and FIG. 4B shows the same in heat-treated (HT) serum, in absence or presence of 10 μM Compound B (circles denote vehicle control; triangles denote compound B), and wherein FIG. 4C) shows bacterial survival after 24 h incubation in serum inoculated with E. coli 25922, (0.9±0.02)×10⁴ CFU/mL and supplemented with 10 μM Compound B, 18 μM lysozyme or 13 μM lactoferrin, as assessed alone and in combinations (C denotes PBS vehicle control, O denoted Compound B, LZ denotes lysozyme, LF denotes lactoferrin; error bars represent standard deviations from the mean);

FIGS. 5A-C present growth kinetics data of serum-resistant K. pneumoniae 1287 in normal (FIG. 5A) or heat-treated (HT) (FIG. 5B) serum, in absence or presence of 10 μM Compound B. Symbols: circles, vehicle control; triangles, Compound B, and FIG. 5C shows bacterial survival after 24 hours incubation in serum inoculated with K. pneumoniae 1287, (1.3±0.08)×10⁴ and supplemented with 10 μM Compound B, 18 μM lysozyme or 13 μM lactoferrin, as assessed alone and in combinations (C denotes PBS vehicle control), O denotes Compound B, LZ denotes lysozyme, LF denotes lactoferrin; error bars represent standard deviations from the mean);

FIGS. 6A-B present growth kinetics assessed by measuring the absorbance at 620 nm of E. coli 25922 (FIG. 6A) and K. pneumoniae 1287 (FIG. 6B) in absence or presence of the 10 μM of Compound B (circles denote vehicle control, triangles denote Compound B; error bars represent standard deviations);

FIGS. 7A-B present mouse peritonitis-sepsis model, wherein FIG. 7A shows survival of neutropenic ICR mice (10/group) infected intraperitoneally with Escherichia coli 25922, 1.2×10⁶ CFU/mouse or Klebsiella pneumoniae 1287, (0.78±0.05)×10⁷ CFU/mouse (left and right, respectively) and treated subcutaneously with Compound B, 1 hour or 1 and 6 hours after inoculation, wherein the right panel, data points represent average from 2 independent experiments (standard deviations were less than 10%), and wherein FIG. 7B shows a variant assay where neutropenic ICR mice (10/group) were infected intraperitoneally with untreated (control) or pretreated E. coli 25922, (1.3±0.283)×10⁶ CFU/mouse or K. pneumoniae 1287, (9.75±0.354). 106 CFU/mouse, and in Compound B-treated groups bacteria were pre-incubated in vitro with 5 μM Compound B for 15 minutes (plotted are the surviving mice after 3 days post-infection);

FIGS. 8A-D present the results obtained for the thigh-infection model, wherein normal mice (8/group) were inoculated intramuscularly with Escherichia coli 25922 (panel a), Klebsiella pneumoniae 1287 (FIG. 8C) or MRSA USA300 10017 (FIG. 7D), and treated subcutaneously 1 hour thereafter (dashed lines represent the inoculums; data points represent the CFU counts obtained after homogenizing the thighs of mice euthanized 24 hours post-treatment), and wherein FIG. 8B shows TNF-α blood levels as determined by ELISA 24 h after E. coli infection in treated, untreated and uninfected mice (Compound B at 12.5 mg/kg body weight; R denotes reference plasma from uninfected mice);

FIGS. 9A-C show evidence for membrane damages to E. coli 25922, wherein FIG. 9A presents time- and dose-dependent data supporting OM permeabilization as evaluated 6 minutes after exposing bacteria to Compound B or PMB in the presence of hydrophobic fluorescent dye NPN, FIG. 9B presents similar data supporting CM depolarization upon pre-incubation of bacteria with potential-sensitive dye (DiSC₃5) and ulteriorly treated with Compound B or PMB, and FIG. 9C presents CM permeabilization data obtained using DNA binder (ethidium bromide) in the presence of Compound B or PMB (data points taken at t=20 minutes; insets show representative kinetics, using 0 and 10 mM Compound B or PMB; positive control (PC) for full depolarization and permeabilization was achieved with C12K7α8 (50 mM) [Rotem, S. et al. FASEB J., 2008, 22, 2652-2661] (FU denotes fluorescence units; triangles denote Compound B, circles denote PMB, squares denote untreated control; error bars=SD);

FIGS. 10A-B present results of simultaneous versus delayed drug exposure assays, wherein E. coli 25922 was exposed in fresh LB culture medium to both Compound B (10 mM) and antibiotic without delay (CT) or after delaying exposure for specified time periods to 0.06 μg/ml rifampin (FIG. 10A) or 4 μg/ml erythromycin (FIG. 10B), whereas CFU counts were determined after additional 3 hours incubation in LB (UC denotes untreated control, CT denotes combined treatment, Rif denotes rifampin; C₁₀O denotes Compound B, Ery denotes erythromycin; dashed line represents inoculum; error bars=SD);

FIG. 11 presents results of a bactericidal kinetic assays conducted in broth versus plasma, wherein the left panels depict time-kill experiments using E. coli 25922 exposed for the specified time periods to Compound B (C₁₀OOC₁₂O; right strips) and rifampin (left strips) or their combination (Grey), and wherein the right panels depict the same experiment where erythromycin substitutes for rifampin (vehicle-treated controls are represented in white columns; dashed line represents the inoculum; asterisk indicates values below detection limit; concentrations: Compound B, 0.6 μM in LB and Human plasma, 10 μM in mouse plasma; Rifampin, 1 μg/ml; Erythromycin, 3 μg/ml; error bars=SD).

FIGS. 12A-C present broth vs. plasma bactericidal kinetics, wherein time-kill studies of E. coli 25922 exposed to vehicle only (denoted by circles), combination of Compound B plus rifampin (denoted by squares), or Compound B plus erythromycin (denoted by triangles) (concentrations: Compound B, 0.6 μM in LB broth and human plasma, 10 μM in mouse plasma; rifampin, 1 μg/ml; erythromycin, 3 μg/ml; error bars=SD); and

FIGS. 13A-B present the results of single versus combination therapy using mouse peritonitis-sepsis model, showing survival kinetics of neutropenic ICR mice (n=10 mice/group) infected intraperitoneally with E. coli 25922 (1.3±0.2×10⁶ CFU/mouse), wherein one hour after infection, mice were treated s.c. with Compound B and/or rifampin (FIG. 13A) or with Compound B and/or erythromycin (FIG. 13B), whereas rifampin was administered orally immediately after inoculation (circles denote vehicle control, inverted triangles denote 20 mg/kg rifampin or 100 mg/kg erythromycin, triangles denote 12.5 mg/kg Compound B, diamonds denote combination of Compound B+rifampin or Compound B+erythromycin).

### DESCRIPTION OF SPECIFIC EMBODIMENTS OF THE INVENTION

The present invention, in some embodiments thereof, relates to non-antibiotic pharmaceutically active compounds, compositions, uses and methods of treatments using the same, and more particularly, to compounds that elicit an improved host-mediated antimicrobial activity, and potentiate antimicrobial drugs against microorganisms including drug-resistant microorganisms.

The principles and operation of the present invention may be better understood with reference to the figures and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

As discussed above, the use of the currently practiced antimicrobial agents and therapies is severely limited, mainly by the development of resistance against these antimicrobial agents. Among the solutions proposed to overcome the current antibiotic deadlock, membrane-active compounds attract a renewed attention for their potential to affect a variety of critical bacterial processes. Because membrane-active compounds are able to target multiple vital bacterial functions simultaneously, they may overcome infections while avoiding many of the known resistance mechanisms. Unlike hydrophobic membrane-active compounds (e.g., dermaseptins) that instigate drastic membrane disruption that, ultimately, may kill bacteria, borderline-hydrophobic membrane-active compounds involved in superficial membrane interactions tend to cause damage that, while repairable, confers a high metabolic cost to bacteria. For instance, the ordered packing of the membrane constituents can be distorted by steric hindrance of bulky membrane-active compounds to a level whereby transient proton leakage occurs, thereby temporarily affecting the transmembrane potential, which is required for vital bioenergetics and transport functions. Although bacteria may be more likely to acquire resistance to a bacteriostatic rather than a bactericidal antibiotic, experimental evidence indicates that, in the presence of a borderline-hydrophobic membrane-active compounds, bacteria were less likely to develop resistance to conventional antibiotics. While this scenario was proposed to sensitize gram-negative bacteria to efflux substrate antibiotics, studies suggested that similar membrane-active compound interactions with the outer membrane might sensitize gram-negative bacteria to low permeability antibiotics. Thus, the present inventors have developed non-bactericidal compounds following structure-activity relationship studies of a synthetic library of polymeric cationic membrane-active compounds. As demonstrated in the Examples section presented below, these compounds exhibit a surprising activity profile, since despite their lack of antimicrobial activity, and their inefficiency in inhibiting gram-negative bacterial proliferation (a consequence of its efflux by RND pumps), the compounds permeabilized their outer membrane to other antibiotics, such as rifampin. Moreover, combination administration of these compounds and rifampin in systemic treatment of infected mice had superior efficacy over individual administration of the drugs, thereby attributing the enhanced in vivo performance of the compounds to increased bioavailability and capacity for outer membrane permeabilization.

In order to further verify the underlying expectancies, the inventors have investigated whether compounds-mediated in vivo outer membrane damage could allow bacterial sensitization to intrinsic factors associated with the antibacterial activity in the infected organism. Some of these factors, such as lysozyme, that damage bacterial cell walls within lysosomal phagocytes and in the serum-soluble form are clearly underexploited for therapeutic purposes. Indeed, the compounds provided herein showed immunopotentiating activity, namely while not being antibiotic per se, these compounds elicited an improved and longer-lasting immune-response in the host organism.

Active Compounds:

According to an aspect of embodiments of the present invention, there is provided a compound selected from the group consisting of:

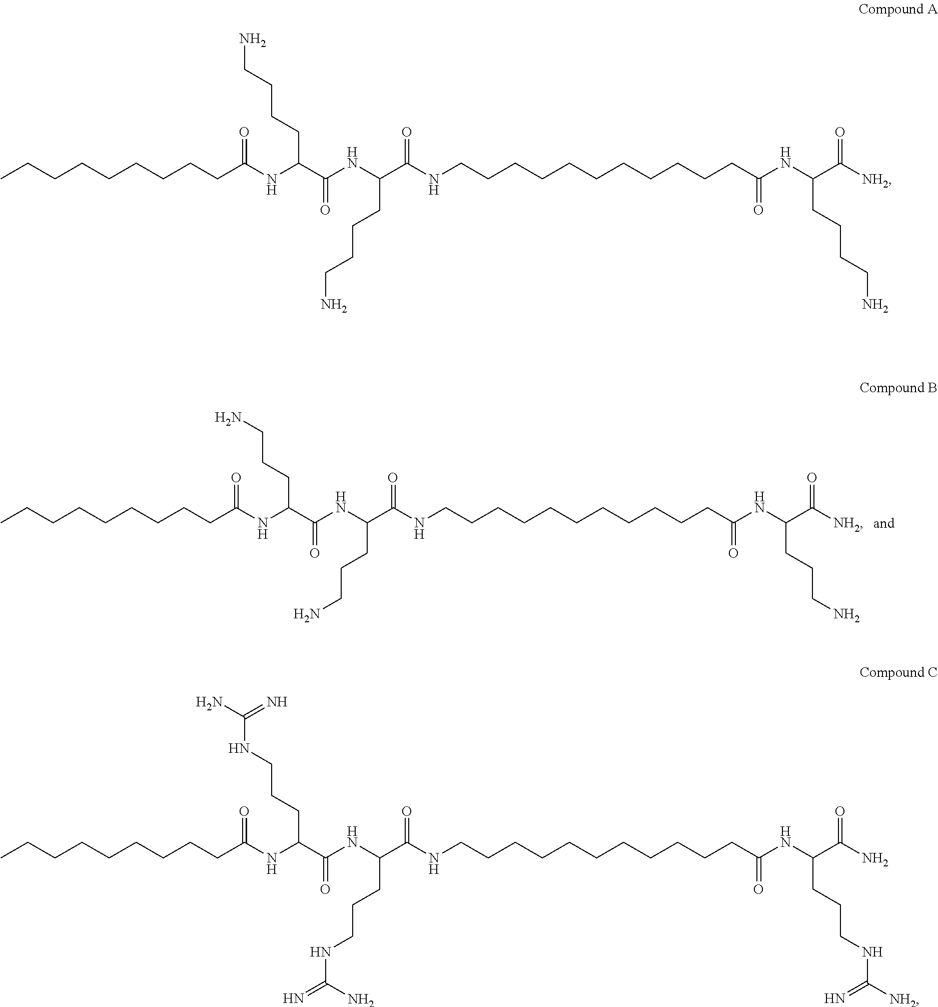 [SMILES: CCCCCCCCCC(=O)NC(CCCCN)C(=O)NC(CCCCN)C(=O)NCCCCCCCCCCCC(=O)NC(CCCCN)C(N)=O ; CCCCCCCCCC(=O)NC(CCCN)C(=O)NC(CCCN)C(=O)NCCCCCCCCCCCC(=O)NC(CCCN)C(N)=O ; "and" ; CCCCCCCCCC(=O)NC(CCCNC(=N)N)C(=O)NC(CCCNC(=N)N)C(=O)NCCCCCCCCCCCC(=O)NC(CCCNC(=N)N)C(N)=O]

whereas the term “compound” encompasses any enantiomer, prodrug, solvate, hydrate and/or pharmaceutically acceptable salt thereof. In some embodiments, the compound is Compound A. In some embodiments, the compound is Compound B. In some embodiments, the compound is Compound C.

As used herein, the term “enantiomer” refers to a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems.

The term “prodrug” refers to an agent, which is converted into the active compound (the active parent drug) in vivo. Prodrugs are typically useful for facilitating the administration of the parent drug. They may, for instance, be bioavailable by oral administration whereas the parent drug is not. A prodrug may also have improved solubility as compared with the parent drug in pharmaceutical compositions. Prodrugs are also often used to achieve a sustained release of the active compound in vivo. An example, without limitation, of a prodrug would be a compound of the present invention, having one or more carboxylic acid moieties, which is administered as an ester (the “prodrug”). Such a prodrug is hydrolyzed in vivo, to thereby provide the free compound (the parent drug). The selected ester may affect both the solubility characteristics and the hydrolysis rate of the prodrug.

The term “solvate” refers to a complex of variable stoichiometry (e.g., di-, tri-, tetra-, penta-, hexa-, and so on), which is formed by a solute (the compound as described herein) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

As used herein, the phrase “pharmaceutically acceptable salt” refers to a charged species of the parent compound and its counter-ion, which is typically used to modify the solubility characteristics of the parent compound and/or to reduce any significant irritation to an organism by the parent compound, while not abrogating the biological activity and properties of the administered compound. A pharmaceutically acceptable salt of a compound as described herein can alternatively be formed during the synthesis of the compound, e.g., in the course of isolating the compound from a reaction mixture or re-crystallizing the compound.

In the context of some of the present embodiments, a pharmaceutically acceptable salt of the compounds described herein may optionally be an acid addition salt comprising at least one basic (e.g., amine and/or guanidine) group of the compound which is in a positively charged form (e.g., wherein the basic group is protonated), in combination with at least one counter-ion, derived from the selected base, that forms a pharmaceutically acceptable salt.

The acid addition salts of the compounds described herein may therefore be complexes formed between one or more basic groups of the compound and one or more equivalents of an acid.

Depending on the stoichiometric proportions between the charged group(s) in the compound and the counter-ion in the salt, the acid additions salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

An example, without limitation, of a pharmaceutically acceptable salt of the compounds presented herein, would be an ammonium cation or guanidinium cation and an acid addition salt thereof. The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

In the context of some embodiments of the present invention, the compounds presented herein are not antimicrobial agents, as they exhibit essentially no antibacterial activity. By “no antibacterial activity” it is meant that the minimal inhibition concentration (MIC) thereof for a particular strain is much higher than the concentration of a compound that is considered an antibiotic with respect to this strain. Further, the MIC of these compounds is notably higher than the concentration required for exerting the desired bacterial sensitization activity, or drug potentiation and/or immunopotentiation activity. As demonstrated below, the compounds presented herein are essentially devoid of an antimicrobial activity against a pathogenic microorganism, as measured in an isolate preparation of the microorganism. In other words, when tested in vitro in a medium that supports the bacteria, but lacks other factors and agents, the compounds were not bactericidal, at least at concentrations below 50 μM, below 40 μM, below 30 μM, below 20 μM, or below 10 μM, namely the compounds exhibited MIC levels higher than 10 μM, higher than 20 μM, higher than 30 μM, higher than 40 μM, or higher than 50 μM.

In the context of some embodiments of the present invention, each of the terms “isolate”, “diagnostic isolate” or “isolate preparation”, refers to a medium that includes the bacterial strain in under investigation which has been isolated from an infected organism, and ingredients that are essential for bacterial proliferation. This isolate is used to test the sensitivity and susceptibility of the bacterium to a given antibiotic agent as a result of direct interaction between the bacterium and the antibiotic agent. In the context of embodiments of the present invention, a diagnostic isolate is a mean by which a decision is made whether to use a specific antibiotic drug against the bacterium in question; typically, an antibiotic agent, which have shown null or low antimicrobial activity in a diagnostic isolate against a tested pathogen isolated from an infected organism, would not be selected for treatment of an infection caused by the tested pathogen. In contrast to an assay conducted in an isolate preparation, a serum or blood sample containing the pathogenic microorganism, includes factors and agents of the immune system and other elements that play a role in an organisms' endogenic antimicrobial defense systems.

Pathogenic Microorganism:

The compounds presented herein are useful in treating a wide range of pathogenic microorganisms, both as immunopotentiating agents and/or potentiating co-drags when working in synergy with antimicrobial agents. As presented hereinbelow, the pathogenic microorganisms are rendered more susceptible to the host's antimicrobial defense systems, or more susceptible to an antimicrobial agent. Herein throughout, the phrase “pathogenic microorganism” is used to describe any microorganism which can cause a disease or disorder in a higher organism, such as mammals in general and a human in particular. The pathogenic microorganism may belong to any family of organisms such as, but not limited to prokaryotic organisms, eubacterium, archaebacterium, gram-negative bacteria, gram-positive bacteria, eukaryotic organisms, yeast, fungi, algae, protozoan, and other parasites. Non-limiting examples of pathogenic microorganism are Plasmodium falciparum and related malaria-causing protozoan parasites, Acanthamoeba and other free-living amoebae, Aeromonas hydrophila, Anisakis and related worms, and further include, but not limited to Acinetobacter baumanii, Ascaris lumbricoides, Bacillus cereus, Brevundimonas diminuta, Campylobacter jejuni, Clostridium botulinum, Clostridium perfringens, Cryptosporidium parvum, Cyclospora cayetanensis, Diphyllobothrium, Entamoeba histolytica, certain strains of Escherichia coli, Eustrongylides, Giardia lamblia, Klebsiella pneumoniae, Listeria monocytogenes, Nanophyetus, Plesiomonas shigelloides, Proteus mirabilis, Pseudomonas aeruginosa, Salmonella species, Salmonella enterica, Serratia odorifera, Shigella, Staphylococcus aureus, Stenotrophomonas maltophilia, Streptococcus, Trichuris trichiura, Vibrio cholerae, Vibrio parahaemolyticus, Vibrio vulnificus and other vibrios, Yersinia enterocolitica, Yersinia pseudotuberculosis and Yersinia kristensenii.

Pharmaceutical Compositions:

In any of the methods and uses described herein, the compounds described herein can be utilized either per se or form a part of a pharmaceutical composition, which further includes a pharmaceutically acceptable carrier, as defined herein. Thus, according to an aspect of some embodiments of the present invention, there is provided a pharmaceutical composition that includes, as an active ingredient, any of the compounds described herein and a pharmaceutically acceptable carrier.

In some embodiments, the pharmaceutical composition is packaged in a packaging material and/or identified in print, in or on the packaging material that the composition is for use in the treatment of a medical condition associated with a pathogenic microorganism in a subject. As demonstrated hereinbelow, the pharmaceutical composition includes the compounds presented herein despite or because the compound is essentially devoid an antimicrobial activity against the pathogenic microorganism in an isolate thereof.

A condition associated with a pathogenic microorganism describes an infectious condition that results from the presence of the microorganism in a subject. The infectious condition can be, for example, a bacterial infection, a fungal infection, a protozoal infection, and the like.

As used herein a “pharmaceutical composition” refers to a preparation of the compounds presented herein, with other chemical components such as pharmaceutically acceptable and suitable carriers and excipients. The purpose of a pharmaceutical composition is to facilitate administration of a compound to an organism.

Hereinafter, the term “pharmaceutically acceptable carrier” refers to a carrier or a diluent that does not cause significant irritation to an organism and does not abrogate the biological activity and properties of the administered compound. Examples, without limitations, of carriers are: propylene glycol, saline, emulsions and mixtures of organic solvents with water, as well as solid (e.g., powdered) and gaseous carriers.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences” Mack Publishing Co., Easton, Pa., latest edition, which is incorporated herein by reference.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more pharmaceutically acceptable carriers comprising excipients and auxiliaries, which facilitate processing of the compounds presented herein into preparations which, can be used pharmaceutically. Proper formulation is dependent upon the route of administration chosen.

According to some embodiments, the administration is effected orally. For oral administration, the compounds presented herein can be formulated readily by combining the compounds with pharmaceutically acceptable carriers well known in the art. Such carriers enable the compounds presented herein to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

The pharmaceutical composition may be formulated for administration in either one or more of routes depending on whether local or systemic treatment or administration is of choice, and on the area to be treated. Administration may be done orally, by inhalation, or parenterally, for example by intravenous drip or intraperitoneal, subcutaneous, intramuscular or intravenous injection, or topically (including ophtalmically, vaginally, rectally, intranasally).

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the compounds presented herein may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For injection, the compounds presented herein may be formulated in aqueous solutions, preferably in physiologically compatible buffers such as Hank's solution, Ringer's solution, or physiological saline buffer with or without organic solvents such as propylene glycol, polyethylene glycol.

For transmucosal administration, penetrants are used in the formulation. Such penetrants are generally known in the art.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compounds doses.

For buccal administration, the compositions may take the form of tablets or lozenges formulated in conventional manner.

For administration by inhalation, the compounds presented herein are conveniently delivered in the form of an aerosol spray presentation (which typically includes powdered, liquefied and/or gaseous carriers) from a pressurized pack or a nebulizer, with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the compounds presented herein and a suitable powder base such as, but not limited to, lactose or starch.

The compounds presented herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the compounds preparation in water-soluble form. Additionally, suspensions of the compounds presented herein may be prepared as appropriate oily injection suspensions and emulsions (e.g., water-in-oil, oil-in-water or water-in-oil in oil emulsions). Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents, which increase the solubility of the compounds presented herein to allow for the preparation of highly concentrated solutions.

Alternatively, the compounds presented herein may be in powder form for constitution with a suitable vehicle, e.g., sterile, pyrogen-free water, before use.

The compounds presented herein may also be formulated in rectal compositions such as suppositories or retention enemas, using, e.g., conventional suppository bases such as cocoa butter or other glycerides.

The pharmaceutical compositions herein described may also comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols.

Pharmaceutical compositions suitable for use in context of the present invention include compositions wherein the active ingredients are contained in an amount effective to achieve the intended purpose. More specifically, a therapeutically effective amount means an amount of compounds presented herein effective to prevent, alleviate or ameliorate symptoms of the disorder, or prolong the survival of the subject being treated.

Determination of a therapeutically effective amount is well within the capability of those skilled in the art, especially in light of the detailed disclosure provided herein.

For any compounds presented herein used in the methods of the present embodiments, the therapeutically effective amount or dose can be estimated initially from activity assays in animals. For example, a dose can be formulated in animal models to achieve a circulating concentration range that induces acceptable or desired activity levels, as determined by activity assays (e.g., the concentration of the test compounds which achieves the desired therapeutic effect). Such information can be used to more accurately determine useful doses in humans.

Toxicity and therapeutic efficacy of the compounds presented herein can be determined by standard pharmaceutical procedures in experimental animals, e.g., by determining the EC50 (the concentration of a compound where 50% of its maximal effect is observed) and the LD50 (lethal dose causing death in 50% of the tested animals) for a subject compound. The data obtained from these activity assays and animal studies can be used in formulating a range of dosage for use in human.

The dosage may vary depending upon the dosage form employed and the route of administration utilized. The exact formulation, route of administration and dosage can be chosen by the individual physician in view of the patient's condition. (See e.g., Fingl et al., 1975, in “The Pharmacological Basis of Therapeutics”, Ch. 1 p.1).

Dosage amount and interval may be adjusted individually to provide plasma levels of the compounds presented herein which are sufficient to maintain the desired therapeutic effects, termed the minimal effective concentration (MEC). The MEC will vary for each preparation, but can be estimated from in vitro data; e.g., the concentration of the compounds necessary to achieve the desired therapeutic effects at least to some extent. Dosages necessary to achieve the MEC will depend on individual characteristics and route of administration. HPLC assays or bioassays can be used to determine plasma concentrations.

Dosage intervals can also be determined using the MEC value. Preparations should be administered using a regimen, which maintains plasma levels above the MEC for 10-90% of the time, preferable between 30-90% and most preferably 50-90%.

Depending on the severity and responsiveness of the chronic condition to be treated, dosing can also be a single periodic administration of a slow release composition described hereinabove, with course of periodic treatment lasting from several days to several weeks or until sufficient amelioration is effected during the periodic treatment or substantial diminution of the disorder state is achieved for the periodic treatment.

The amount of a composition to be administered will, of course, be dependent on the subject being treated, the severity of the affliction, the manner of administration, the judgment of the prescribing physician, etc. Compositions of the present invention may, if desired, be presented in a pack or dispenser device, such as an FDA (the U.S. Food and Drug Administration) approved kit, which may contain one or more unit dosage forms containing the active ingredient. The pack may, for example, comprise metal or plastic foil, such as, but not limited to a blister pack or a pressurized container (for inhalation). The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert. Compositions comprising a compound according to the present embodiments, formulated in a compatible pharmaceutical carrier may also be prepared, placed in an appropriate container, and labeled for treatment of an indicated condition or diagnosis, as is detailed hereinabove.

The conversion of effective amounts found in laboratory research animals is afforded by experimental procedures and by conversion rules, typically based on the organism's body surface area [see, for example, Nair, A. B. et al., “A simple practice guide for dose conversion between animals and human”, Journal of Basic and Clinical Pharmacy, 2016, 7(2), pp. 27-31]. The most common instance is rodent studies wherein the dosages mentioned are applicable to either rats or mice; and wherein there exists the need to calculate the human equivalent dosage (HED). A commonly used formula is as follows: HED (mg/kg)=Animal Dose (mg/kg)×[Animal Kₘ/Human Kₘ], wherein human Kₘ=37, mouse Kₘ=3 and rat Kₘ=6. For animal weights outside the working weight range, or for species not included in the literature, an alternative method is available for calculating the HED. In these cases the following formula can be used: HED=Animal dose (mg/kg)×[animal weight (kg)÷human weight (kg)]. Additional information is readily available in the literature, such as the “Guidance for Industry Estimating the Maximum Safe Starting Dose in Initial Clinical Trials for Therapeutics in Adult Healthy Volunteers”, available from the Office of Training and Communications Division of Drug Information, HFD-240 Center for Drug Evaluation and Research Food and Drug Administration 5600 Fishers Lane Rockville, Md. 20857, USA.

In the Examples section that follows below, any effective mount of any of the ingredients, which have been determined in mice, can be readily converted into HED using the abovementioned conversions.

Immunopotentiation:

In the context of embodiments of the present invention, the term “immunopotentiation” refers to the accentuation of an immune response by the administration of an exogenous substance (e.g., an adjuvant).

In some embodiments of the present invention, the pharmaceutical compositions, methods and uses presented herein, the concentration of the active compound provided herein in the subject (e.g., blood levels), also referred to herein as the post-administration amount thereof, is an immunopotentiating amount of the compound.

In the context of embodiments of the present invention, the term “immunopotentiating amount”, refers to a concentration of a substance that is sufficient to affect the activity of endogenous antimicrobial defense systems of the organism, i.e. the immune system, so as to overcome an infectious pathogen or be more effective in overcoming an infectious pathogen. Without being bound by any particular theory, it is said that the compounds presented herein may act as immunostimulants, or as agents that assist, elicit, promote, enhance or stimulate an immune response against a pathogen. This immunopotentiating activity exhibited by the compounds provided herewith is demonstrated in the Examples section that follows below, which is attributed, without being bound by any particular theory, to the interaction of the compounds with membranes of the pathogen, thereby either rendering the pathogen more susceptible to cell killing factors, or more conspicuous to the immune system.

This exceptional approach, with respect to any therapeutic agent, and particularly with respect to an agent that is used to combat infectious diseases and medical conditions associated with pathogenic microorganisms, which encourages the administration of an agent that has been shown not to be active in a diagnostic isolate, is applicable to the compounds presented herein as well as to any of the compounds previously presented in WO/2006/035431, WO/2008/132737, WO/2008/132738, WO/2009/090648, WO/2008/072242 and WO/2011/016043, which are incorporated herein by reference.

According to some embodiments of the present invention, methods of treatment, uses and pharmaceutical compositions, which are based on the compounds presented herein as their sole active ingredient, are based on the antimicrobial defense mechanisms of the infected organism, and on the ability of the administered compound to immunopotentiate these defense mechanisms. Hence, in some embodiments, the methods of treatment, uses and pharmaceutical compositions presented herein are essentially devoid of an antimicrobial agent.

Combination Therapy:

In any of the compositions, methods and uses described herein, the compounds can be utilized in combination with other agents useful in the treatment of the medical condition, disease or disorder, and/or in inducing or promoting a therapeutically desired activity. In the context of embodiments of the present invention, being primarily directed at treating medical conditions associated with the presence of a pathogenic microorganism in a subject, the additional agents are antimicrobial agents, and possibly other immunostimulants and the like.

The phrase “antimicrobial agent”, as used herein, excludes the compounds provided herein according to the embodiments of the present invention, and encompasses all other antimicrobial agents. According to the definition of microorganism presented hereinabove, the phrase “antimicrobial agent” encompasses antibiotic agents (also referred to herein as antibiotic) as well as anti-fungal, anti-protozoan, anti-parasitic agents and like.

According to some embodiments, the antimicrobial agent is an antibiotic agent. In general, but without being bound to any particular theory, the mechanism of the antimicrobial activity of an antimicrobial agent, according to the embodiments of the present invention, is different that the mechanism of the activity of the compounds provided herein.

According to some embodiments, the compounds presented herein render any antimicrobial agent more potent against any bacterial strain, due to the generality of their mode of action, which involves targeting the microorganisms' membranes. Thus, the antimicrobial agent being co-administered with the compound in a combination therapy method and composition, may be a broad-spectrum antibiotic agent, or a species-specific antibiotic agent. The pathogenic microorganism may be tolerant (resistant) to the selected antimicrobial agent, yet in a combination therapy regime, the microorganism will be rendered sensitive again (re-sensitized) to the antimicrobial agent as a result of the activity of the compound. Furthermore, an antimicrobial agent that is known not to be active against a specific family or species of microorganism, may be rendered effective due to the cooperation and synergism exhibited in the combined treatment. For these reasons an antimicrobial agent can be used in a combined therapy regime in a lower concentration compared to its effective amount when used alone. The antimicrobial agent can be inactive, or be less effective for any reason, or be highly effective as a standalone mono-treatment, yet in the combined therapy regime it will be co-administered at lower concentrations than a comparable standalone mono-treatment.

Non-limiting examples of antimicrobial agents that are suitable for use in this context of the present invention include, without limitation, mandelic acid, 2,4-dichlorobenzenemethanol, 4-[bis(ethylthio)methyl]-2-methoxyphenol, 4-epi-tetracycline, 4-hexylresorcinol, 5,12-dihydro-5,7,12,14-tetrazapentacen, 5-chlorocarvacrol, 8-hydroxyquinoline, acetarsol, acetylkitasamycin, acriflavin, alatrofloxacin, ambazon, amfomycin, amikacin, amikacin sulfate, aminoacridine, aminosalicylate calcium, aminosalicylate sodium, aminosalicylic acid, ammoniumsulfobituminat, amorolfin, amoxicillin, amoxicillin sodium, amoxicillin trihydrate, amoxicillin-potassium clavulanate combination, amphotericin B, ampicillin, ampicillin sodium, ampicillin trihydrate, ampicillin-sulbactam, apalcillin, arbekacin, aspoxicillin, astromicin, astromicin sulfate, azanidazole, azidamfenicol, azidocillin, azithromycin, azlocillin, aztreonam, bacampicillin, bacitracin, bacitracin zinc, bekanamycin, benzalkonium, benzethonium chloride, benzoxonium chloride, berberine hydrochloride, biapenem, bibrocathol, biclotymol, bifonazole, bismuth subsalicylate, bleomycin antibiotic complex, bleomycin hydrochloride, bleomycin sulfate, brodimoprim, bromochlorosalicylanilide, bronopol, broxyquinolin, butenafine, butenafine hydrochloride, butoconazol, calcium undecylenate, candicidin antibiotic complex, capreomycin, carbenicillin, carbenicillin disodium, carfecillin, carindacillin, carumonam, carzinophilin, caspofungin acetate, cefacetril, cefaclor, cefadroxil, cefalexin, cefalexin hydrochloride, cefalexin sodium, cefaloglycin, cefaloridine, cefalotin, cefalotin sodium, cefamandole, cefamandole nafate, cefamandole sodium, cefapirin, cefapirin sodium, cefatrizine, cefatrizine propylene glycol, cefazedone, cefazedone sodium salt, cefazolin, cefazolin sodium, cefbuperazone, cefbuperazone sodium, cefcapene, cefcapene pivoxil hydrochloride, cefdinir, cefditoren, cefditoren pivoxil, cefepime, cefepime hydrochloride, cefetamet, cefetamet pivoxil, cefixime, cefmenoxime, cefmetazole, cefmetazole sodium, cefminox, cefminox sodium, cefmolexin, cefodizime, cefodizime sodium, cefonicid, cefonicid sodium, cefoperazone, cefoperazone sodium, ceforanide, cefoselis sulfate, cefotaxime, cefotaxime sodium, cefotetan, cefotetan disodium, cefotiam, cefotiam hexetil hydrochloride, cefotiam hydrochloride, cefoxitin, cefoxitin sodium, cefozopran hydrochloride, cefpiramide, cefpiramide sodium, cefpirome, cefpirome sulfate, cefpodoxime, cefpodoxime proxetil, cefprozil, cefquinome, cefradine, cefroxadine, cefsulodin, ceftazidime, cefteram, cefteram pivoxil, ceftezole, ceftibuten, ceftizoxime, ceftizoxime sodium, ceftriaxone, ceftriaxone sodium, cefuroxime, cefuroxime axetil, cefuroxime sodium, cetalkonium chloride, cetrimide, cetrimonium, cetylpyridinium, chloramine T, chloramphenicol, chloramphenicol palmitate, chloramphenicol succinate sodium, chlorhexidine, chlormidazole, chlormidazole hydrochloride, chloroxylenol, chlorphenesin, chlorquinaldol, chlortetracycline, chlortetracycline hydrochloride, ciclacillin, ciclopirox, cinoxacin, ciprofloxacin, ciprofloxacin hydrochloride, citric acid, clarithromycin, clavulanate potassium, clavulanate sodium, clavulanic acid, clindamycin, clindamycin hydrochloride, clindamycin palmitate hydrochloride, clindamycin phosphate, clioquinol, cloconazole, cloconazole monohydrochloride, clofazimine, clofoctol, clometocillin, clomocycline, clotrimazol, cloxacillin, cloxacillin sodium, colistin, colistin sodium methanesulfonate, colistin sulfate, cycloserine, dactinomycin, danofloxacin, dapsone, daptomycin, daunorubicin, DDT, demeclocycline, demeclocycline hydrochloride, dequalinium, dibekacin, dibekacin sulfate, dibrompropamidine, dichlorophene, dicloxacillin, dicloxacillin sodium, didecyldimethylammonium chloride, dihydrostreptomycin, dihydrostreptomycin sulfate, diiodohydroxyquinolin, dimetridazole, dipyrithione, dirithromycin, DL-menthol, D-menthol, dodecyltriphenylphosphonium bromide, doxorubicin, doxorubicin hydrochloride, doxycycline, doxycycline hydrochloride, econazole, econazole nitrate, enilconazole, enoxacin, enrofloxacin, eosine, epicillin, ertapenem sodium, erythromycin, erythromycin estolate, erythromycin ethyl succinate, erythromycin lactobionate, erythromycin stearate, ethacridine, ethacridine lactate, ethambutol, ethanoic acid, ethionamide, ethyl alcohol, eugenol, exalamide, faropenem, fenticonazole, fenticonazole nitrate, fezatione, fleroxacin, flomoxef, flomoxef sodium, florfenicol, flucloxacillin, flucloxacillin magnesium, flucloxacillin sodium, fluconazole, flucytosine, flumequine, flurithromycin, flutrimazole, fosfomycin, fosfomycin calcium, fosfomycin sodium, framycetin, framycetin sulphate, furagin, furazolidone, fusafungin, fusidic acid, fusidic acid sodium salt, gatifloxacin, gemifloxacin, gentamicin antibiotic complex, gentamicin c1a, gentamycin sulfate, glutaraldehyde, gramicidin, grepafloxacin, griseofulvin, halazon, haloprogine, hetacillin, hetacillin potassium, hexachlorophene, hexamidine, hexetidine, hydrargaphene, hydroquinone, hygromycin, imipenem, isepamicin, isepamicin sulfate, isoconazole, isoconazole nitrate, isoniazid, isopropanol, itraconazole, josamycin, josamycin propionate, kanamycin, kanamycin sulphate, ketoconazole, kitasamycin, lactic acid, lanoconazole, lenampicillin, leucomycin A1, leucomycin A13, leucomycin A4, leucomycin A5, leucomycin A6, leucomycin A7, leucomycin A8, leucomycin A9, levofloxacin, lincomycin, lincomycin hydrochloride, linezolid, liranaftate, 1-menthol, lomefloxacin, lomefloxacin hydrochloride, loracarbef, lymecyclin, lysozyme, mafenide acetate, magnesium monoperoxophthalate hexahydrate, mecetronium ethylsulfate, mecillinam, meclocycline, meclocycline sulfosalicylate, mepartricin, merbromin, meropenem, metalkonium chloride, metampicillin, methacycline, methenamin, methyl salicylate, methylbenzethonium chloride, methylrosanilinium chloride, meticillin, meticillin sodium, metronidazole, metronidazole benzoate, mezlocillin, mezlocillin sodium, miconazole, miconazole nitrate, micronomicin, micronomicin sulfate, midecamycin, minocycline, minocycline hydrochloride, miocamycin, miristalkonium chloride, mitomycin c, monensin, monensin sodium, morinamide, moxalactam, moxalactam disodium, moxifloxacin, mupirocin, mupirocin calcium, nadifloxacin, nafcillin, nafcillin sodium, naftifine, nalidixic acid, natamycin, neomycin a, neomycin antibiotic complex, neomycin C, neomycin sulfate, neticonazole, netilmicin, netilmicin sulfate, nifuratel, nifuroxazide, nifurtoinol, nifurzide, nimorazole, niridazole, nitrofurantoin, nitrofurazone, nitroxolin, norfloxacin, novobiocin, nystatin antibiotic complex, octenidine, ofloxacin, oleandomycin, omoconazol, orbifloxacin, ornidazole, ortho-phenylphenol, oxacillin, oxacillin sodium, oxiconazole, oxiconazole nitrate, oxoferin, oxolinic acid, oxychlorosene, oxytetracycline, oxytetracycline calcium, oxytetracycline hydrochloride, panipenem, paromomycin, paromomycin sulfate, pazufloxacine, pefloxacin, pefloxacin mesylate, penamecillin, penicillin G, penicillin G potassium, penicillin G sodium, penicillin V, penicillin V calcium, penicillin V potassium, pentamidine, pentamidine diisetionate, pentamidine mesilas, pentamycin, phenethicillin, phenol, phenoxyethanol, phenylmercuriborat, PHMB, phthalylsulfathiazole, picloxydin, pipemidic acid, piperacillin, piperacillin sodium, pipercillin sodium-tazobactam sodium, piromidic acid, pivampicillin, pivcefalexin, pivmecillinam, pivmecillinam hydrochloride, policresulen, polymyxin antibiotic complex, polymyxin B, polymyxin B sulfate, polymyxin B 1, polynoxylin, povidone-iodine, propamidin, propenidazole, propicillin, propicillin potassium, propionic acid, prothionamide, protiofate, pyrazinamide, pyrimethamine, pyrithion, pyrrolnitrin, quinoline, quinupristin-dalfopristin, resorcinol, ribostamycin, ribostamycin sulfate, rifabutin, rifampicin, rifamycin, rifapentine, rifaximin, ritiometan, rokitamycin, rolitetracycline, rosoxacin, roxithromycin, rufloxacin, salicylic acid, secnidazol, selenium disulphide, sertaconazole, sertaconazole nitrate, siccanin, sisomicin, sisomicin sulfate, sodium thiosulfate, sparfloxacin, spectinomycin, spectinomycin hydrochloride, spiramycin antibiotic complex, spiramycin b, streptomycin, streptomycin sulphate, succinylsulfathiazole, sulbactam, sulbactam sodium, sulbenicillin disodium, sulbentin, sulconazole, sulconazole nitrate, sulfabenzamide, sulfacarbamide, sulfacetamide, sulfacetamide sodium, sulfachlorpyridazine, sulfadiazine, sulfadiazine silver, sulfadiazine sodium, sulfadicramide, sulfadimethoxine, sulfadoxine, sulfaguanidine, sulfalene, sulfamazone, sulfamerazine, sulfamethazine, sulfamethazine sodium, sulfamethizole, sulfamethoxazole, sulfamethoxazol-trimethoprim, sulfamethoxypyridazine, sulfamonomethoxine, sulfamoxol, sulfanilamide, sulfaperine, sulfaphenazol, sulfapyridine, sulfaquinoxaline, sulfasuccinamide, sulfathiazole, sulfathiourea, sulfatolamide, sulfatriazin, sulfisomidine, sulfisoxazole, sulfisoxazole acetyl, sulfonamides, sultamicillin, sultamicillin tosilate, tacrolimus, talampicillin hydrochloride, teicoplanin A2 complex, teicoplanin A2-1, teicoplanin A2-2, teicoplanin A2-3, teicoplanin A2-4, teicoplanin A2-5, teicoplanin A3, teicoplanin antibiotic complex, telithromycin, temafloxacin, temocillin, tenoic acid, terbinafine, terconazole, terizidone, tetracycline, tetracycline hydrochloride, tetracycline metaphosphate, tetramethylthiuram monosulfide, tetroxoprim, thiabendazole, thiamphenicol, thiaphenicol glycinate hydrochloride, thiomersal, thiram, thymol, tibezonium iodide, ticarcillin, ticarcillin-clavulanic acid mixture, ticarcillin disodium, ticarcillin monosodium, tilbroquinol, tilmicosin, tinidazole, tioconazole, tobramycin, tobramycin sulfate, tolciclate, tolindate, tolnaftate, toloconium metilsulfat, toltrazuril, tosufloxacin, triclocarban, triclosan, trimethoprim, trimethoprim sulfate, triphenylstibinsulfide, troleandomycin, trovafloxacin, tylosin, tyrothricin, undecoylium chloride, undecylenic acid, vancomycin, vancomycin hydrochloride, viomycin, virginiamycin antibiotic complex, voriconazol, xantocillin, xibornol and zinc undecylenate.

In some embodiments, the antimicrobial agent is an antibiotic. Exemplary antibiotics include, but are not limited to oxacillin, piperacillin, penicillin G, ciprofloxacin, erythromycin, tetracycline, gentamicin and methicillin. These antibiotics are known to be associated with emergence of resistance thereto.

Methods of Treatment:

Treating a condition associated with a pathogenic microorganism describes means for preventing, reducing, ameliorating or abolishing symptoms of the infectious or other medical condition in a subject. The treatment is effected typically by inhibiting the growth and/or eradicating the pathogenic microorganism in a subject in need thereof.

The compound presented herein may be used in a monotherapy method of treatment, wherein the compound is administered as an immunopotentiating agent to elicit, improve, enhance the effectiveness of, and/or stimulate the subject's immune system. In such methods of treatment, the subject's own antimicrobial defense systems do the actual killing of the pathogen, while the compound plays an adjuvant role, namely an additive that enhances the effectiveness of the medical treatment.

Thus, according to an aspect of embodiments of the resent invention, there is provided a method of treating a medical condition associated with a pathogenic microorganism in a subject, which is effected by administering an immunopotentiating amount of the compound provided herein to the subject.

According to some embodiments of the present invention, the phrase “immunopotentiating amount” refers to the “therapeutically effective amount” of the compound in the context of monotherapy; thus, in some embodiments, the method is effected without the use of an antimicrobial agent, and the “immunopotentiating amount” describes an amount of the compound being administered, which will relieve to some extent one or more of the symptoms of the condition being treated.

As demonstrated in the Examples section that follows, the compounds presented were found highly effective when administered together with an antimicrobial agent in eradicating a range of pathogenic bacteria including bacteria resistant to the antimicrobial agent or resistant to other antimicrobial agents. The compounds were shown capable of re-sensitizing bacteria which became resistant to an antibiotic, such that when the same antibiotic is re-used, it effectively eradicates the bacteria. The compounds are also capable of preventing the emergence of resistance, when used in combination with an antibiotic, in microorganisms that are expected to develop resistance to the antibiotic.

The compounds are therefore highly useful in treating conditions associated with resistant bacteria, by (i) being effective when administered in combination with an antimicrobial treatment that would otherwise not be effective; (ii) being effective in preventing an emergence of resistance to an antimicrobial agent, when administered in combination with the antimicrobial agent; and (iii) being effective in re-sensitizing a microorganism to an antimicrobial agent, upon an antimicrobial treatment that resulted in emergence of resistance to the antimicrobial agent used.

Thus, according to one aspect of the present invention there is provided a method of treating a medical condition associated with a pathogenic microorganism in a subject. In some embodiments, the method is effected by co-administering to the subject a therapeutically effective amount of an antimicrobial agent, and co-administering to the subject a therapeutically effective amount of the compound presented herein, wherein:

the effective amount of the antimicrobial agent is lower than a therapeutically effective amount of the antimicrobial agent when administered alone, in monotherapy without the compound, and

the effective amount of the compound is a potentiating amount thereof with respect to the antimicrobial agent.

According to another aspect of the present invention there is provided a method of treating a medical condition associated with a pathogenic microorganism and further associated with an emergence of antimicrobial resistance in a subject still suffering from that medical condition after being treated with an antimicrobial agent. The method is effected by administering to that subject, following the treatment with the antimicrobial agent and the emergence of antimicrobial resistance to the antimicrobial agent, a re-sensitizing amount of the compound as described and exemplified herein, thereby re-sensitizing the microorganism to the antimicrobial agent and treating the medical condition.

The method is further effected by administering to the subject a therapeutically effective amount of the antimicrobial agent. In essence, the antimicrobial agent is re-administered (administered again after the microorganism(s) developed resistance) to the subject, with the distinction that the pathogenic microorganism is now re-sensitized towards the antimicrobial agent by the compound.

According to some embodiments, the two active ingredients, namely the antimicrobial agent and the compound, can be administered concomitantly or the antimicrobial agent can be administered to the subject subsequent to administration of the compound, after the pathogenic microorganism has been re-sensitized by the antimicrobial re-sensitizing compound.

When administered subsequently, the antimicrobial agent can be administered less than 10 minutes, 20 minutes, 30 minutes, 1 hour, 2 hours, 3 hours, 4 hours, 5 hours, 6 hours, 7 hours, 8 hours, 9 hours, 10 hours, 12 hours, 24 hours, and longer, after administration of the compound. In some embodiments, the compound is administered prior to the administration of the antimicrobial agent, following the above timing regimen.

The phrase “antimicrobial re-sensitizing activity”, as used herein in the context of the compounds according to the embodiments presented herein, defines a characteristic of the compound which is related to three entities, namely (i) the compound, (ii) an antimicrobial agent, and (iii) a microorganism which became or may become resistant to the antimicrobial agent in the sense that the microorganism is no longer sensitive to the antimicrobial agent. Thus, the existence on an antimicrobial re-sensitizing activity allows the compound to endow potency to, to increase the potency of, potentiate, or re-potentiate the antimicrobial agent against the microorganism by sensitizing or re-sensitizing the microorganism to the antimicrobial agent.

By “re-sensitizing”, it is meant that a microorganism that was sensitive (susceptible) to a treatment with antimicrobial agent and became resistant to such a treatment, is turned again to be sensitive (susceptible) to such a treatment.

As used herein, the phrases “potentiating amount”, “sensitizing amount”, or “re-sensitizing amount” describes a therapeutically effective amount of the compound, which is sufficient to render the corresponding antimicrobial agent potent, therapeutically effective, and/or sufficient to reverse the emerged resistance towards the antimicrobial agent. In some embodiments, these phrases describe a therapeutically effective amount of the compound which is sufficient to reverse, or prevent, the emergence of resistance in the pathogenic microorganism causing the medical condition.

In the context of the present embodiments, when pertaining to the antimicrobial agent, the phrase “therapeutically effective amount” refers to an amount of an antimicrobial agent being co-administered and/or re-administered, which will relieve to some extent one or more of the symptoms of the condition being treated by being at a level that is harmful to the target microorganism(s), namely a bactericidal level or otherwise a level that inhibits the microorganism growth or eradicates the microorganism.

It should be noted herein that, with respect to the compound according to embodiments of the present invention, a potentiating, sensitizing or re-sensitizing amount, is a specific therapeutically effective amount in the sense that a potentiating, sensitizing or re-sensitizing amount is not expected to directly harm to the target microorganism(s) when used alone, without the presence of an antimicrobial agent.

It should be noted herein that, with respect to the antimicrobial agent a therapeutically effective amount thereof is lower than the therapeutically effective amount thereof when used alone as an antimicrobial agent against the pathogenic microorganism. The antimicrobial agent may be not effective at all, poorly effective or highly effective, nonetheless, its therapeutically effective amount would be higher than its therapeutically effective amount when used in combination with the compound. These drug-compound interaction via the target microorganism and the host, is referred to as a synergistic therapeutic effect.

Thus, according to some embodiments of the invention, due to the re-sensitizing amount of the compound, the therapeutically effective amount of the antimicrobial agent is lower than the therapeutically effective amount of this antimicrobial agent with respect to the microorganism to be eradicated if/when the antimicrobial agent is administered by itself per-se.

The efficacy of an antimicrobial agent is oftentimes referred to in minimal inhibitory concentration units, or MIC units. A MIC is the lowest concentration of an antimicrobial agent, typically measured in micro-molar (μM) or micrograms per milliliter (μg/ml) units, or mg of the antimicrobial agent per kg of subject's weight, that can inhibit the growth of a microorganism after a period of incubation, typically 24 hours. MIC values are used as diagnostic criteria to evaluate resistance of microorganisms to an antimicrobial agent, and for monitoring the activity of an antimicrobial agent in question. MICs are determined by standard laboratory methods, as these are described and demonstrated in the Examples section that follows. Standard laboratory methods typically follow a standard guideline of a reference body such as the Clinical and Laboratory Standards Institute (CLSI), British Society for Antimicrobial Chemotherapy (BSAC) or The European Committee on Antimicrobial Susceptibility Testing (EUCAST). In clinical practice, the minimum inhibitory concentrations are used to determine the amount of antibiotic agent that the subject receives as well as the type of antibiotic agent to be used.

Accordingly, there is provided a method of re-sensitizing a pathogenic microorganism to an antimicrobial agent, following a treatment of the pathogenic microorganism with the antimicrobial agent and a subsequent emergence of a resistance of the pathogenic microorganism to the antimicrobial agent. The method is effected by contacting the pathogenic microorganism with a re-sensitizing amount of the compound(s) described herein.

According to some embodiments of the method of re-sensitizing a pathogenic microorganism presented hereinabove, contacting the microorganism with the compound is effected by administering the re-sensitizing amount of the compound to a subject having a medical condition associated with the microorganism and further associated with an emergence of antimicrobial resistance in this subject following treatment with an antimicrobial agent. The re-sensitizing method can be further be effected by contacting the pathogenic microorganism with the antimicrobial agent, subsequent to or concomitant with the re-sensitization thereof by the compound detailed herein.

According to other embodiments of the method of re-sensitizing a pathogenic microorganism presented hereinabove, administering the compound is followed by administering the antimicrobial agent to the subject. According to embodiments of the present invention, and as stated hereinabove, the antimicrobial agent can be re-administered concomitant with or subsequent to the administration of the compound.

In any of the methods described herein, the compound and/or the antimicrobial agent can be administered as a part of a pharmaceutical composition, which further comprises a pharmaceutical acceptable carrier, as detailed hereinbelow. The carrier is selected suitable to the selected route of administration. The compound and/or the antimicrobial agent can be administered via any administration route, including, but not limited to, orally, by inhalation, or parenterally, for example, by intravenous drip or intraperitoneal, subcutaneous, intramuscular or intravenous injection, or topically (including ophtalmically, vaginally, rectally, intranasally). In some embodiments, the methods are effected by oral, rectal or intraperitoneal administration, by inhalation, or subcutaneous injection.

According to another aspect of the present invention, there is provided a use of a compound as presented herein, in the manufacture of a medicament for treating a medical condition associated with a pathogenic microorganism. The medicament may be used for treating a medical condition associated with a pathogenic microorganism, and further associated with an emergence of antimicrobial resistance in a subject having the medical condition and treated with an antimicrobial agent. According to some embodiments, the medicament is used alone, or in combination with an antimicrobial agent, which is selected such that when a re-sensitizing amount of the compound is used, the re-sensitizing amount being substantially lower than a therapeutically effective amount of the compound with respect to the pathogenic microorganism, as described herein. As in some other aspects presented herein, and according to some embodiments, the compound can be used in combination with the antimicrobial agent, which can then be administered concomitant with or subsequent to administering the compound.

Accordingly, there is provided a use of a compound as described herein in the manufacture of a medicament for re-sensitizing a pathogenic microorganism to an antimicrobial agent following a treatment of the pathogenic microorganism with the antimicrobial agent and a subsequent emergence of a resistance of the pathogenic microorganism to the antimicrobial, wherein a re-sensitizing amount of the compound is used, the re-sensitizing amount being lower than a therapeutically effective amount of the compound with respect to the pathogenic microorganism. Also in this aspect and according to some embodiments, the compound can be used in combination with the antimicrobial agent, which can then be administered concomitant with or subsequent to administering the compound.

A Kit:

In accordance with aspects of the present invention, the compounds presented herein are directed at uses in combination therapy with antimicrobial agents, and as further presented, the two active ingredients may be administered concomitantly or sequentially as separate compositions. Hence, there is an advantage in providing the health-care provider or the self-administering subject a kit, as described below, which will include all the required compositions in one package.

Thus, according to yet another aspect of the present invention, there is provided a pharmaceutical kit which includes inside a packaging material a compound as described herein and an antimicrobial agent being individually packaged. The kit can then be labeled according to its intended use, such as for treating a medical condition associated with a pathogenic microorganism, and may further be associated with an emergence of antimicrobial resistance in a subject having the medical condition and treated with an antimicrobial agent, and/or for re-sensitizing a pathogenic microorganism to an antimicrobial agent, following a treatment of the pathogenic microorganism with the antimicrobial agent and a subsequent emergence of a resistance of the pathogenic microorganism to the antimicrobial.

As used herein the term “about” refers to ±10%.

The terms “comprises”, “comprising”, “includes”, “including”, “having” and their conjugates mean “including but not limited to”. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the phrases “substantially devoid of” and/or “essentially devoid of” in the context of a certain substance, refer to a composition that is totally devoid of this substance or includes less than about 5, 1, 0.5 or 0.1 percent of the substance by total weight or volume of the composition. Alternatively, the phrases “substantially devoid of” and/or “essentially devoid of” in the context of a process, a method, a property or a characteristic, refer to a process, a composition, a structure or an article that is totally devoid of a certain process/method step, or a certain property or a certain characteristic, or a process/method wherein the certain process/method step is effected at less than about 5, 1, 0.5 or 0.1 percent compared to a given standard process/method, or property or a characteristic characterized by less than about 5, 1, 0.5 or 0.1 percent of the property or characteristic, compared to a given standard.

The term “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The words “optionally” or “alternatively” are used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the terms “process” and “method” refer to manners, means, techniques and procedures for accomplishing a given task including, but not limited to, those manners, means, techniques and procedures either known to, or readily developed from known manners, means, techniques and procedures by practitioners of the chemical, material, mechanical, computational and digital arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1

### Materials and Experimental Methods

Compounds' Synthesis:

The compounds presented herein were synthesized by the solid phase method following methodologies disclosed in WO/2006/035431, WO/2008/132737, WO/2008/132738, WO/2009/090648, WO/2008/072242 and WO/2011/016043, all of which are incorporated by reference as if fully set forth herein. Briefly, the compounds were synthesized while applying the Fmoc active ester chemistry on a fully automated, programmable peptide synthesizer (Applied Biosystems 433A). After cleavage from the resin, the crude product was extracted with 30% acetonitrile in water and purified by RP-HPLC (Alliance Waters), so as to obtain a chromatographic homogeneity higher than 95%. HPLC runs were typically performed on C18 columns (Vydac, 250 mm×4.6 or 10 mm) using a linear gradient of acetonitrile in water (1% per minute), both solvents containing 0.1% trifluoroacetic acid. The purified compounds were subjected to mass spectrometry (ZQ Waters) and NMR analyses to confirm their composition and stored as a lyophilized powder at −20° C. Prior to being tested, fresh solutions were prepared in water, vortexed, sonicated, centrifuged and then diluted in the appropriate medium.

Solution Organization:

Organization of the compounds in aqueous solution was assessed by light-scattering measurements in phosphate buffered saline (PBS; 10 mM Na₂HPO₄, 150 mM NaCl, pH=7.0). Hemolytic assays were performed as described elsewhere, using fresh mouse red blood cells.

Pathogens:

The following strains were used for the studies below: Escherichia coli strain ML-35p (American Type Culture Collection [ATCC]; Manassas, Va.), E. coli strain ATCC 25922, Klebsiella pneumoniae clinical isolate 1287, K. pneumoniae carbapenemase 2-producing strain, Salmonella enterica serovar Choleraesuis (ATCC 7308), S. enterica serovar Typhimurium (ATCC 14028), and Pseudomonas aeruginosa clinical isolates 11662 and 11816. The following gram-positive species was investigated: methicillin-resistant Staphylococcus aureus clinical isolate USA300 10017 (a gift from Dr. Gili Regev-Yochay, Infectious Disease Unit, Sheba Medical Center, Israel). Additional Escherichia coli strains tested include β-lactamase producer 35218; clinical isolate strains 14182, 14384, and U16327; and two K12 isogenic mutants, AG100 and AG100A. Unless otherwise stated, all bacterial cultures were grown overnight in Luria-Bertani broth (LB).

Minimal Inhibitory Concentrations:

Minimal inhibitory concentrations (MICs) were determined as previously described. Briefly, MIC is defined as the lowest drug concentration that induced a 100% inhibition of proliferation at standard growth conditions of a given bacterium. MICs were determined by microdilution susceptibility testing in 96-well plates (by Nunc) using inocula of 10⁵ bacteria per ml. Cell populations were evaluated by optical density measurements at 620 nm and were calibrated against a set of standards. Hundred (100) μl of a bacterial suspension were added to 100 μl of culture medium (control) or to 100 μl of culture medium containing various tested compound concentrations in 2-fold serial dilutions. Inhibition of proliferation was determined by optical density measurements at 620 nm after an incubation period of 24 hours at 37° C. Data were obtained from ≥2 independent experiments performed in duplicate.

Membrane Permeabilization:

Membrane permeabilization was assessed using E. coli ML-35p as described elsewhere, to monitor the ability of the compounds to perturb the outer and/or cytoplasmic membranes. Reported data were obtained from ≥2 independent experiments performed in duplicate.

Serum Assays:

Serum survival assays were performed using fresh serum from normal mice or human serum from the Israel Blood Bank; samples were pooled and stored in aliquots at −80° C. until use. Time-dependent killing was determined in a final volume of 125 μL consisting of 112.5 μL of serum containing either a tested compound, egg white lysozyme (Amresco), lactoferrin (Vivinal lactoferrin FD; DMV International, Delhi, N.Y.), anti-murine C5/C5a antibody (25 μg/mL; ab194637; Abcam), or their combination, as specified. These solutions were supplemented with 12.5 μL of bacteria suspended in PBS at the desired concentration. After the specified time points of exposure, cultures were subjected to serial 10-fold dilutions in saline (0.85% NaCl) and plated for bacterial enumeration after incubation at 37° C. for an additional 24 hours. In heat-treated experiments, sera were incubated at 56° C. for 30 minutes for protein inactivation. Data were obtained from 3 independent experiments.

In Vivo Studies:

In vivo studies described below were performed using male ICR mice (mean weight [±SD], 23±2 g) obtained from Envigo Laboratories (Rehovot, Israel).

Toxicity:

The maximum tolerated dose was determined after a single-dose subcutaneous administration of the tested compounds, using 8, 8, and 2 mice, respectively. Animals were inspected for adverse effects for 6 hours after injection. Mortality was monitored for 7 days thereafter. Pharmacokinetic studies were performed as described elsewhere.

Mouse Peritonitis-Sepsis Model:

A mouse peritonitis-sepsis model was created as described elsewhere. Infection was obtained after intraperitoneal injection of bacteria from a logarithmic-phase culture. Infected mice were treated subcutaneously. The doses were selected to allow comparison with the reference compound and to remain below the maximum tolerated dose after double-dose administration. Typically, treating this infection model by using ciprofloxacin or imipenem yields a survival frequency of 80-100%, as reported elsewhere. Briefly, mouse peritonitis sepsis model for in vivo studies were performed using male ICR mice (23 6 2 g) obtained from Envigo Laboratories (Rehovot, Israel). Mice were rendered neutropenic by intraperitoneal injection of cyclophosphamide (150 and 100 mg/kg on day 0 and day 3, respectively) and the procedure confirmed to result in severe neutropenia by day 4, at which time infection was induced. Infection was obtained by intraperitoneal injection of a logarithmic phase culture of E. coli 25922 (1.3±0.2×10⁶ CFU permouse in 0.3 ml PBS). Immediately thereafter, mice (10/group) were treated orally with rifampin (0.25 ml distilled water containing 0.45±0.02 mg/mouse); the tested compound and erythromycin were administered subcutaneously, each at a single dose (12.5 and 100 mg/kg, respectively) an hour after inoculation. Infection control mice were injected with the PBS vehicle. Mouse survival was monitored for up to 7 days after treatment. Statistical analyses were performed by paired Student's t test, at α=0.05.

Mouse Thigh Infection Model:

Mouse thigh infection model was afforded from normal ICR mice, which were inoculated intramuscularly and treated subcutaneously 1 hour thereafter with a tested compound or polymyxin B (PMB; Sigma-Aldrich) as described elsewhere. P values were calculated using a 1-tailed t test (assuming unequal variance). A P value of <0.05 is considered statistically significant.

Immunosorbent Assays:

Enzyme-linked immunosorbent assays were performed using blood samples collected from mice 24 hours after infection and centrifuged (at 1000×g for 5 minutes). Murine tumor necrosis factor α levels were determined in accordance with the manufacturer instructions (PeproTech).

Electron Microscopy:

For high-resolution scanning electron microscopy (SEM), silicon chips and sample treatment were prepared as described elsewhere with the following minor modifications: Mid-logarithmic phase E. coli 25922 at 1×10⁷ CFU/ml was centrifuged for 5 minutes at 15,000 g. Pellets were washed twice with PBS, resuspended in the same buffer containing 0 or 10 mM of the tested compound, and incubated for 15 minutes at 37 □ with shaking. Thereafter the samples were carbon coated by graphite sputtering and studied by SEM (Ultra Plus; Carl Zeiss, Jena, Germany). Microscope gun intensity was set to 1 kV and the working distance to 3 mm.

Outer-Membrane Damage Assay:

1-N-phenylnaphthylamine (NPN) uptake (manifested as fluorescence) reflects outer-membrane damage because normally bacteria are able to exclude hydrophobic substances. As described elsewhere, E. coli 25922 was grown to the mid-logarithmic phase of growth (OD=0.4-0.6 at 600 nm), centrifuged, and resuspended in 5 mM HEPES buffer containing 5 mM glucose to OD 0.5. Next, 50 ml of NPN solution (0.2 mM) was added to every milliliter bacteria suspension. Using black 96-well plates, 190 ml of bacteria suspension was mixed with 10 ml of test compound at the desired concentration and the fluorescence immediately monitored (excitation 360 nm, emission 460 nm) for up to 10 min. Data were obtained from at least 2 independent experiments performed in triplicate.

Cytoplasmic Membrane Depolarization:

Cytoplasmic membrane depolarization measurements were assessed by monitoring the displacement of 3,39-dipropylthiadicarbocyanine iodide (DiSC₃5), a lipophilic potentiometric dye. Mid-logarithmic phase E. coli 25922 at OD 0.6 nm was centrifuged for 5 min at 15,000 g. The pellet was washed twice with 5 mM HEPES containing 5 mM glucose and 2 mM EDTA before addition of DiSC₃5 (4 mM) and quenching at room temperature in the dark for 1 hour. KCl was then added (100 mM) and the suspension incubated overnight (4° C.). One hundred eighty microliters of this bacterial suspension was placed in a black 96-well plate, and fluorescence was recorded until signal stabilization (excitation 620 nm, emission 680 nm). Thereafter 20 ml of the tested compound was added and the fluorescence recorded for up to 30 minutes at 37° C. with shaking. Data were obtained from at least 2 independent experiments performed in duplicate.

Ethidium Bromide Permeability Assay:

Ethidium bromide permeability assays were performed as follows: Overnight cultures were adjusted to 1.0 OD (600 nm) and centrifuged for 5 minutes at 15,000 g. Pellets were washed twice with PBS containing 0.5% glucose, resuspended, and incubated for 10 minutes at 37° C. with shaking. A 180-ml suspension was mixed in a black 96-well plate with 25 ml of the tested compound and ethidiumbromide (1 μg/ml), and the fluorescence was recorded (excitation 535 nm, emission 590 nm) for up to 30 minutes at 37° C. with shaking. Data were obtained from at least 2 independent experiments.

Time-Kill Kinetics:

Time-kill kinetics data were determined using 100 μl of bacterial suspension (10⁶ CFU/ml), which was added to 900 ml LB containing none of or specified concentrations of the tested compound, antibiotic, and combinations thereof. After the specified exposure time points (37° C. under shaking), aliquots were formed, subjected to serial 10-fold dilutions in saline (0.85% NaCl), and plated for bacterial enumeration after additional 24 hours incubation at 37° C. Time-kill experiments in human and mouse plasma were performed as described elsewhere. The effects of drug delay were assessed in LB, except that bacteria were pre-incubated with the tested compound or antibiotic for the specified time points, centrifuged, and resuspended in fresh LB-containing antibiotic or the tested compound, respectively, and incubated for 3 hours before CFU enumeration.

Statistical Analyses:

Statistical analyses were performed using a paired t test with an a level of 0.05.

### Example 2

### Sensitization of Pathogens

Active Compounds:

The compounds (see, Scheme 1 below), according to the present embodiments, comprise fatty acid (acyl) residues, positively charged residues (lysine, ornithine and/or arginine) and ω-amino-fatty acid residues, were prepared according to the general procedure described elsewhere.

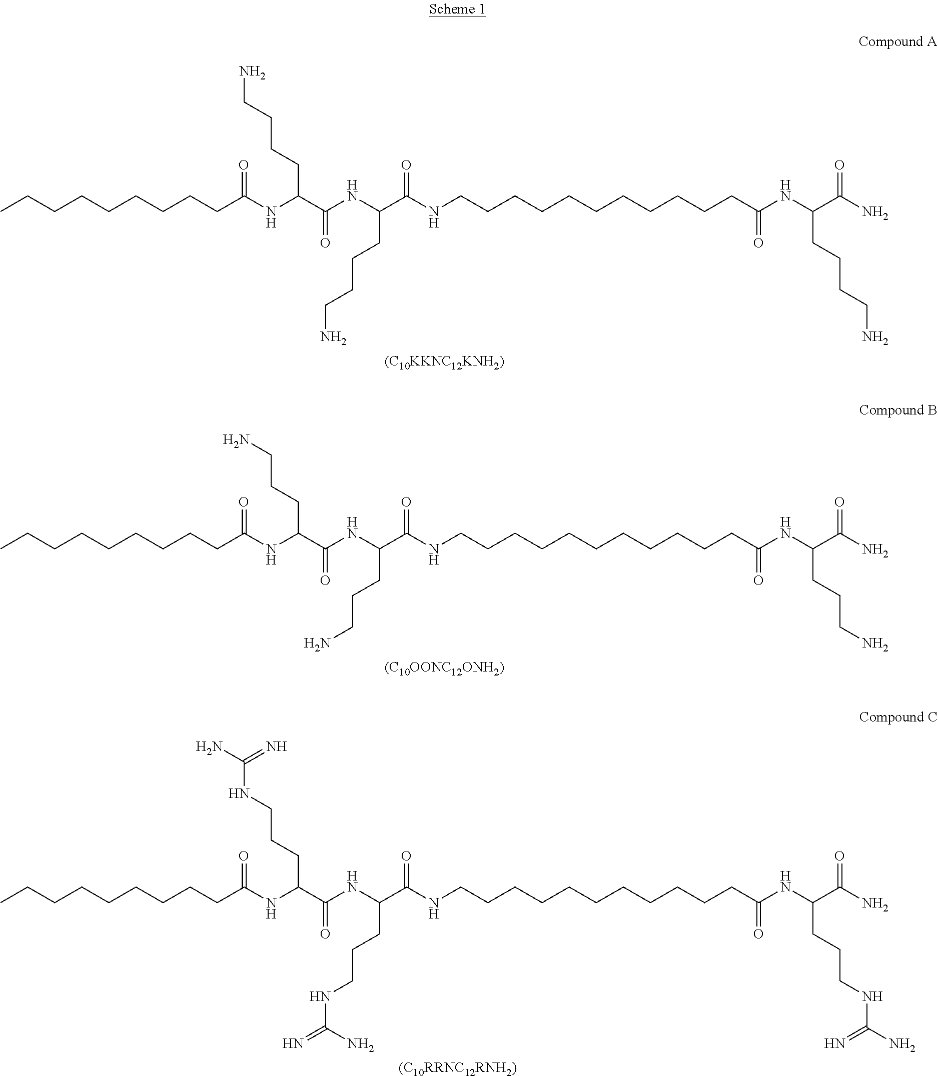

As described hereinabove, the compounds described herein have unique features that enable to use these compounds as immunopotentiating agents, antimicrobial agent potentiating agents or microbial re-sensitization agents. The present embodiments further encompass methods and compositions using any enantiomers, prodrugs, solvates, hydrates and/or pharmaceutically acceptable salts of the compounds described herein.

Antibiotic and Outer Membrane Activity, and Blood Levels:

To address the need for alternatives to antibiotics, gram-negative bacilli were sensitize to innate antibacterial protagonists. Initial work aimed to identify compounds with improved bioavailability. In particular, compounds that maintain membrane-active properties while being devoid of antibiotic activity per se (i.e., unable to kill bacteria on its own but able to induce killing via a third agent); such compounds would expand the sensitivity spectrum to include low permeability antimicrobials, yet they would lack antibiotic activity, which might give them mechanistic advantages (e.g., in avoiding ambiguity as to “who is doing what” among paired drugs) during combination experiments.

Activity assay results are presented in Table 1 below, wherein: Ec, Escherichia coli; Kp, Klebsiella pneumoniae; Pa, Pseudomonas aeruginosa; SC, Salmonella enterica serovar Choleraesuis; ST, Salmonella enterica serovar Typhimurium; Q, charge at physiological pH; H, hydrophobicity determined by the percentage of acetonitrile required for elution during high-performance liquid chromatography when coinjected into a C18 column; critical aggregation concentration (CAC), determined by light scattering in phosphate-buffered saline; fifty percent lethal concentration (LC50) defined as the minimum concentration of the tested compounds required to induce hemolysis of 50% of mouse red blood cells (1%) after 3 hours of incubation in phosphate-buffered saline. The mean value (±SD) is shown for Compound C; Minimum inhibitory concentration (MIC) of compounds required to inhibit bacterial proliferation after overnight incubation.

TABLE 1

| | | | | | MIC (μM), by Bacterium species | | | | | | | |
|---|---|---|---|---|---|---|---|---|---|---|---|---|
| | | H | CAC | LC₅₀ | Ec | Ec | Kp | Kp | SC | ST | Pa | Pa |
| Compound | Q | (%) | (μM) | (μM) | ML35p | 25922 | 1287 | C2 | 7308 | 14028 | 11662 | 11816 |
| A | 3 | 44 | >100 | >100 | >50 | >50 | >50 | >50 | >50 | >50 | >50 | >50 |
| B | 3 | 43 | >100 | >100 | >50 | >50 | >50 | >50 | >50 | >50 | 50 | 50 |
| C | 3 | 46 | >100 | 80 ± 1 | 12.5 | 25 | 50 | >50 | >50 | >50 | 50 | 50 |

As can be seen in Table 1 that summarizes relevant biophysical attributes of the compounds, the data indicate that, while Compound B is less hydrophobic than Compound A, it exhibited similar features in terms of organization in solution and inactivity on bacteria (the minimal bactericidal concentration was >50 μM for E. coli and K. pneumoniae) or on erythrocytes (the concentration required to induce 50% hemolysis was extremely high (i.e., 50% lethal concentration, >100 μM)). In contrast, Compound C displayed a higher hydrophobicity, coupled with some hemolytic and antibacterial capacities. Combined, these data provide evidence for the consistent inefficiency of Compound B in significantly affecting growth of gram-negative bacteria.

Additional support for this view was obtained by comparing the compounds' susceptibility to damage gram-negative bacteria membranes, using the E. coli mutant ML-35p. Widely described in the literature, the mutant was considered herein as representative of all gram-negative bacteria, rather than a specific species. This mutant is constitutive for cytoplasmic β-galactosidase, lacks lactose permease, and expresses a plasmid-encoded periplasmic β-lactamase. Two chromogenic reporter molecules (nitrocefin and 2-nitrophenyl β-d-galactopyranoside, respectively, absorbing at 486 and 420 nm) were used to monitor permeabilization of the outer membrane and/or cytoplasmic membrane in a single assay.

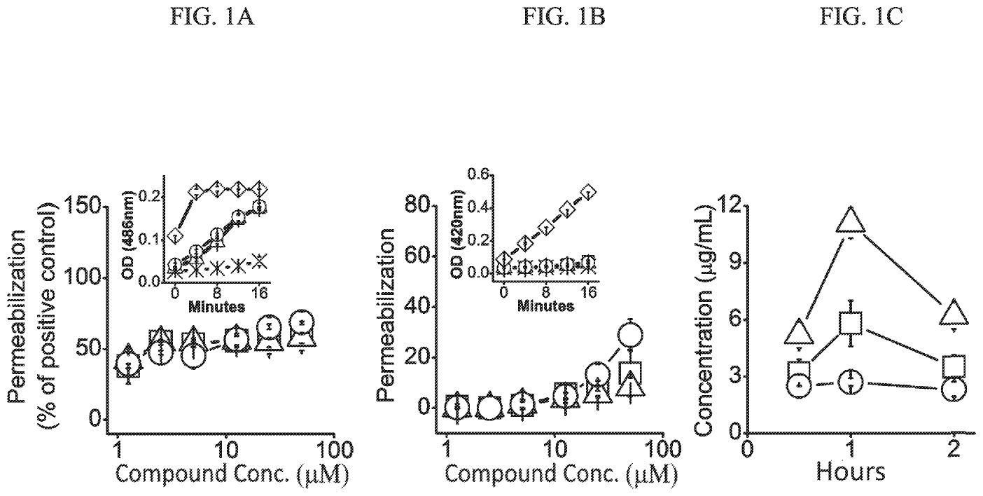

FIGS. 1A-C present the results of the membrane damage and bioavailability assessments, showing dose-dependent permeabilization of the outer (FIG. 1A) and cytoplasmic (FIG. 1B) membranes of the Escherichia coli mutant ML-35p, as determined in buffer, 16 minutes after addition of the compounds presented herein, wherein dermaseptin (25 μM) was used as positive control, representing full permeabilization, and the insets show representative kinetics at 12.5 μM, and further showing plasma concentrations (FIG. 1C) determined by liquid chromatography-mass spectrometry after subcutaneous administration (12.5 mg/kg body weight) to ICR mice (squares denote Compound A, triangles denote Compound B, circles denote Compound C, Xs denote vehicle control and diamonds denote dermaseptin; data are for 2 mice/time point; error bars represent standard deviations).

As can be seen in FIG. 1A, Compound A and Compound B were similarly potent in inducing outer membrane permeabilization and similarly unable to permeabilize the cytoplasmic membrane at least up to approximately 10 μM (FIG. 1B), whereas Compound C exhibited somewhat higher tendency for cytoplasmic membrane damaging.

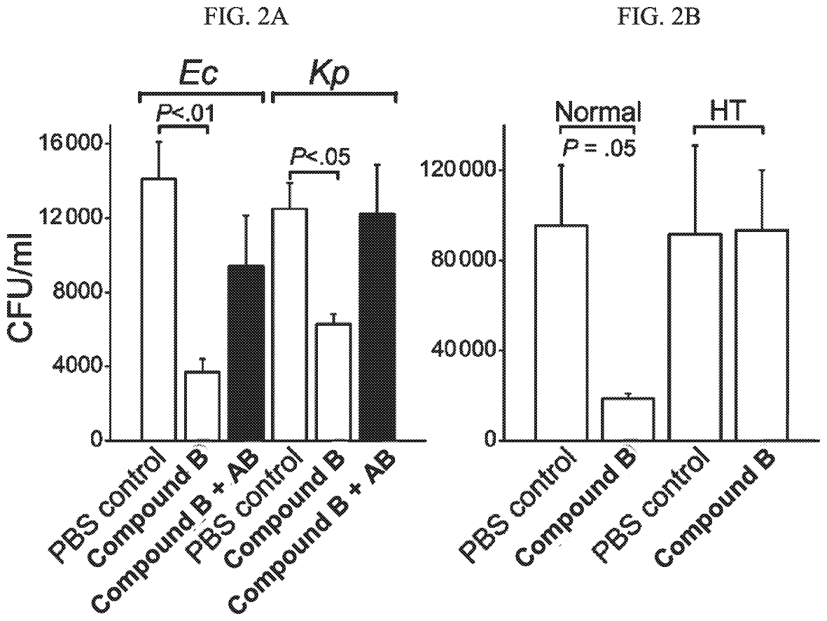

The comparative plots shown in FIG. 1C displays the plasma concentrations of the compounds presented herein, as determined by quantitative liquid chromatography-mass spectrometry following subcutaneous administration (doses were 12.5 mg/kg body weight each) to ICR mice. While the compounds presented herein achieved circulating levels of magnitudes comparable to those of classical antibiotics, their concentrations correlated with their hydrophobicity, predicting a comparatively low bioavailability for Compound C, whose maximal extractable levels were lower than 3 μM. Similarly, the data predicted a relatively higher bioavailability of Compound B, whose extractable levels consistently were higher than 5 μM throughout at least 2 hours after administration. This value may bare importance in subsequent studies (such as those summarized in FIG. 2B). Subcutaneous administration of the highest tested dose (20 mg/kg) was well tolerated, as no adverse effects were observed for any of the compounds presented herein (i.e., the maximal tolerated dose is estimated to be more than 20 mg/kg body weight).

Collectively, these findings support the view that, while Compound B is as efficient as dermaseptin in altering outer membrane permeability, it is at least as inefficient in affecting gram-negative bacteria growth but promises improved pharmacokinetics. This compound was deemed to have the highest potential to address the study's aim and was therefore selected to undergo further characterization.

Activity in Serum:

FIGS. 2A-B present results of antibacterial activity assays of mouse serum, wherein FIG. 2A shows bacterial survival in 80% serum inoculated with Escherichia coli 25922 (Ec) (0.9±0.2)×10³ CFU/mL or Klebsiella pneumoniae 1287 (Kp) (1.08±0.21) □ 10³ CFU/mL, treated with PBS vehicle (control) or 10 μM Compound B and incubated for 3 h (37 □) in absence or presence of anti-complements C5/C5a mouse antibody (AB), and FIG. 2B shows bacterial survival under roughly similar conditions (i.e., after 3 h incubation in 80% serum) when the serum was obtained 1 hour after subcutaneous administration of the tested compound as described in FIG. 1C, followed by E. coli 25922 inoculation and culture as in FIG. 2A (plot also shows a duplicated sample subjected to heat-treatment (HT); error bars represent standard deviations from the mean).

Reportedly, the bactericidal activity of polymyxins against E. coli, as observed in serum at sub-MIC conditions, was mediated by complement proteins. This prompted the study of the antibacterial properties of Compound B in serum. As can be seen in FIGS. 2A-B, the surprising findings indicates that, in mouse serum, Compound B effectively inhibited growth of serum-resistant gram-negative bacteria (74% and 48% inhibition of E. coli and K. pneumoniae, respectively), whereas inhibition diminished when serum was supplemented with an antibody directed against the complements C5/C5a (FIG. 2A) or when saturated with high inocula (data not shown). To validate this activity, the inventors also determined bacterial survival in mouse serum obtained 1 hour after subcutaneous administration of Compound B (the serum concentration was assumed to be more than 5 μM, according to FIG. 1C). The subsequent inoculation and culture were as described in FIG. 2A, while additionally, a duplicated sample was subjected to heat treatment.

As shown in FIG. 2B, the compound-containing serum also exhibited growth inhibitory activity (80% inhibition; P=0.05). The fact that this inhibition was antagonized by heat treatment substantiated the fold-dependent proteinaceous nature of the antibacterial factor, be it complement or another factor(s). This experiment, therefore, joins the previous finding in suggesting that the compounds presented herein have the capacity to recruit some component(s) of the immune system, as host defense peptides (HDPs) might do.

Since various proteins other than antibodies were implicated in observable serum antibacterial properties, the inventors further explored the possible role of non-antibody proteins by exposing bacteria to Compound B in the presence of lysozyme, which is known to damage bacterial cell walls by catalyzing peptidoglycan degradation. Lysozyme is less effective on gram-negative bacteria because their peptidoglycan is less accessible, owing to the outer membrane barrier. The experimental strategy, therefore, aimed to exploit this fact, predicting that lysozyme activity will increase if the compounds presented herein increase the outer membrane permeability (as per FIG. 1A). Results of the following studies, performed in broth medium and in serum, support this notion.

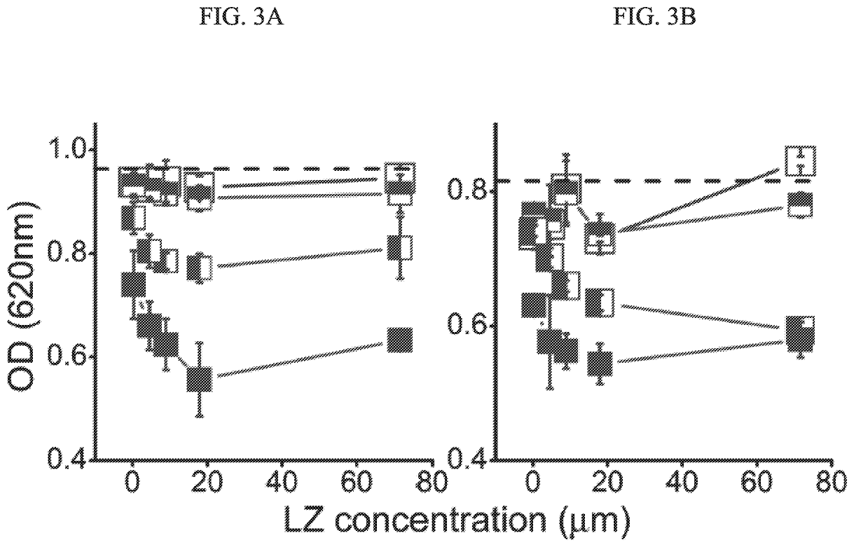

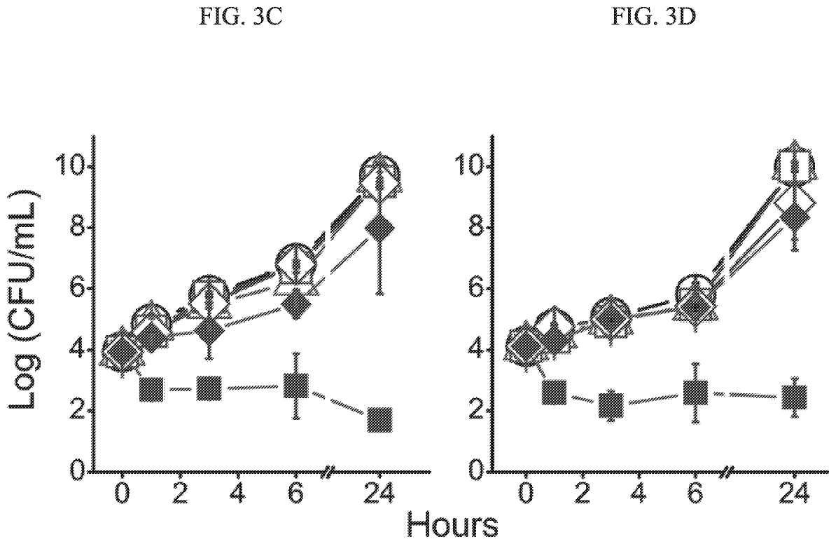

Checkerboard-type experiments exposing bacteria to lysozyme and/or to Compound B in growth medium revealed a dose-dependent synergistic growth inhibition of E. coli and K. pneumoniae in the presence of both drugs combined but not individually, as shown in FIGS. 3A-D. Experiments were performed in the absence of Compound B and in the presence of 2.5, 5, and 10 μM of Compound B, combined with LZ at the specified concentrations. Inhibition was determined after 24 hours of incubation.

FIGS. 3A-D present evidence of synergism of Compound B and lysozyme (LZ) in broth and serum, wherein FIG. 3A and FIG. 3B show the results of the checkerboard assay for bacterial growth inhibition in broth medium containing a mean inoculum (±SD) of 1.1×10⁴±0.05×10⁴ colony-forming units (CFU)/mL of Escherichia coli 25922 (FIG. 3A) or 1.2×10⁴±0.08□10⁴ CFU/mL of Klebsiella pneumoniae 1287 (FIG. 3B), and wherein FIG. 3C and FIG. 3D show the survival of serum-resistant E. coli 25922 and K. pneumoniae 1287 in fresh mouse serum supplemented with 10 μM of Compound B, 18 μM of LZ, or 13 μM of lactoferrin (LF) alone, combination of Compound B and LZ, or combination of Compound B and LF (empty squares denote LZ, top-filled squares denote 2.5 μM Compound B plus LZ, left-filled squares denote 5 μM Compound B plus LZ, full squares denote 10 μM Compound B plus LZ, circles denote vehicle control, triangles denote 10 μM Compound B, empty diamonds denote LF and full diamonds denote 10 μM Compound B plus LF; error bars represent standard deviations from the mean).

As can be seen in FIG. 3C and FIG. 3D, synergism persisted in serum, indicating that, under conditions where lysozyme or Compound B were virtually inactive, the lysozyme-supplemented serum became bactericidal in presence of Compound B. As can be seen in FIG. 3A and FIG. 3D, this synergism was much more potent in serum, as evident from E. coli counts. While the difference in growth was negligible between medium and serum (from 4 to 9.8 log colony-forming units (CFU)/mL and from 4 to 9.7 log CFU/mL, respectively), comparison of the samples treated with 10 μM Compound B plus 18 μM lysozyme to those that were untreated revealed a much greater difference in growth (from 4 to 9 log CFU/mL and from 4 to 1.7 log CFU/mL, respectively). This large difference reflects the fact that additional antibacterial factors (including endogenous lysozyme and complement) were implicated in serum. Albeit, no significant sensitization was observed with lactoferrin, another host defense antimicrobial protein (FIG. 3C and FIG. 3D), possibly hinting to a limitation imposed by size, since lactoferrin is more than 5-fold larger than lysozyme.

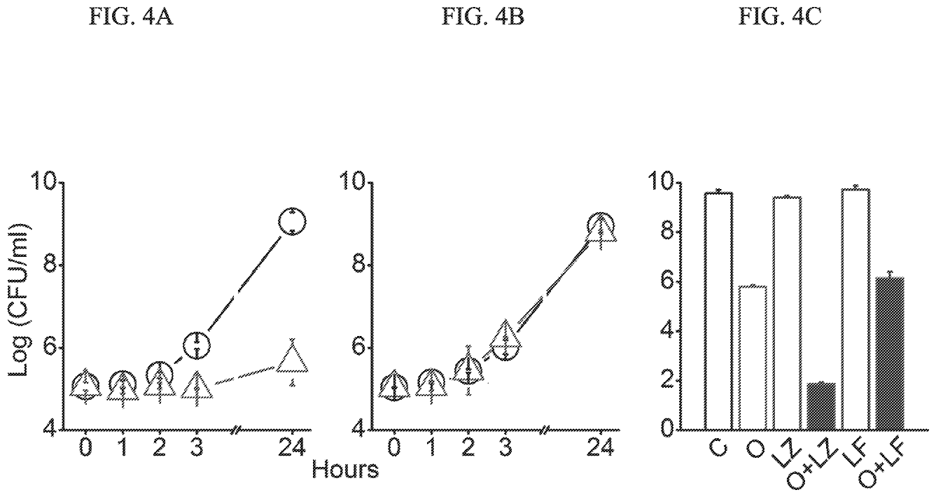

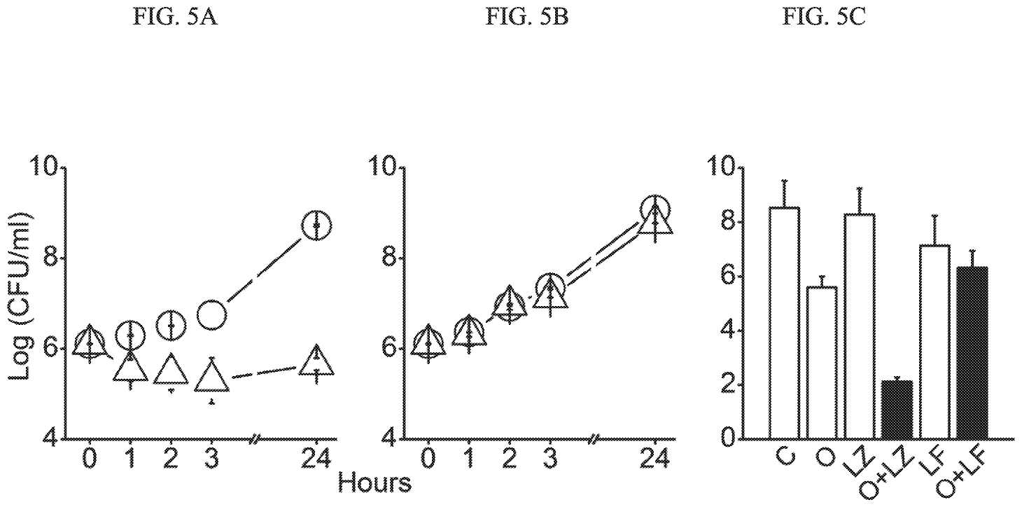

Synergism between human serum components and PMB was previously observed in gram-negative bacteria both in growth medium and serum. The mouse serum antibacterial properties in the presence of Compound B were readily replicated in human serum, as shown in FIGS. 4A-C and FIGS. 5A-C.

FIGS. 4A-C show antibacterial properties of human serum, wherein FIG. 4A shows growth kinetics of serum-resistant Escherichia coli 25922 in normal serum and FIG. 4B shows the same in heat-treated (HT) serum, in absence or presence of 10 μM Compound B (circles denote vehicle control; triangles denote compound B), and werein FIG. 4C) shows bacterial survival after 24 h incubation in serum inoculated with E. coli 25922, (0.9±0.02)×10⁴ CFU/mL and supplemented with 10 μM Compound B, 18 μM lysozyme or 13 μM lactoferrin, as assessed alone and in combinations (C denotes PBS vehicle control, O denoted Compound B, LZ denotes lysozyme, LF denotes lactoferrin; error bars represent standard deviations from the mean).

FIGS. 5A-C present growth kinetics data of serum-resistant K. pneumoniae 1287 in normal (FIG. 5A) or heat-treated (HT) (FIG. 5B) serum, in absence or presence of 10 μM Compound B. Symbols: circles, vehicle control; triangles, Compound B, and FIG. 5C shows bacterial survival after 24 hours incubation in serum inoculated with K. pneumoniae 1287, (1.3±0.08)×10⁴ and supplemented with 10 μM Compound B, 18 μM lysozyme or 13 μM lactoferrin, as assessed alone and in combinations (C denotes PBS vehicle control), O denotes Compound B, LZ denotes lysozyme, LF denotes lactoferrin; error bars represent standard deviations from the mean).

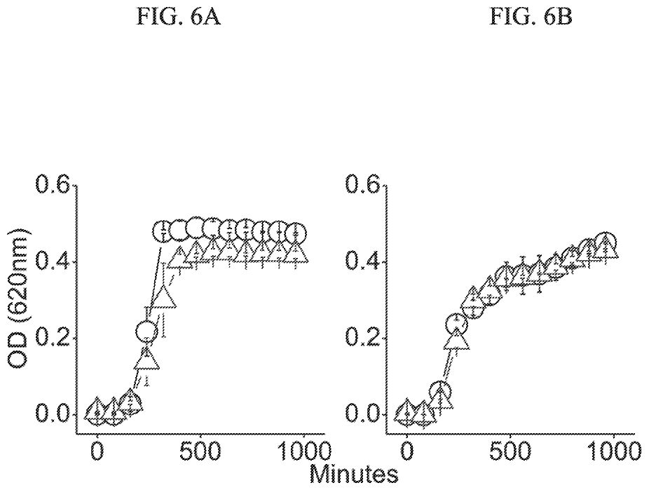

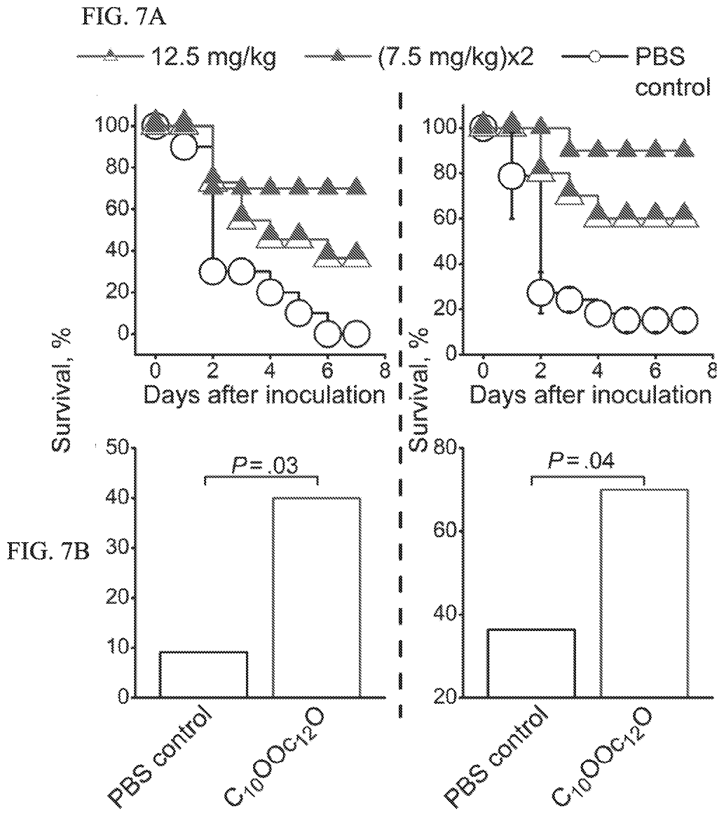

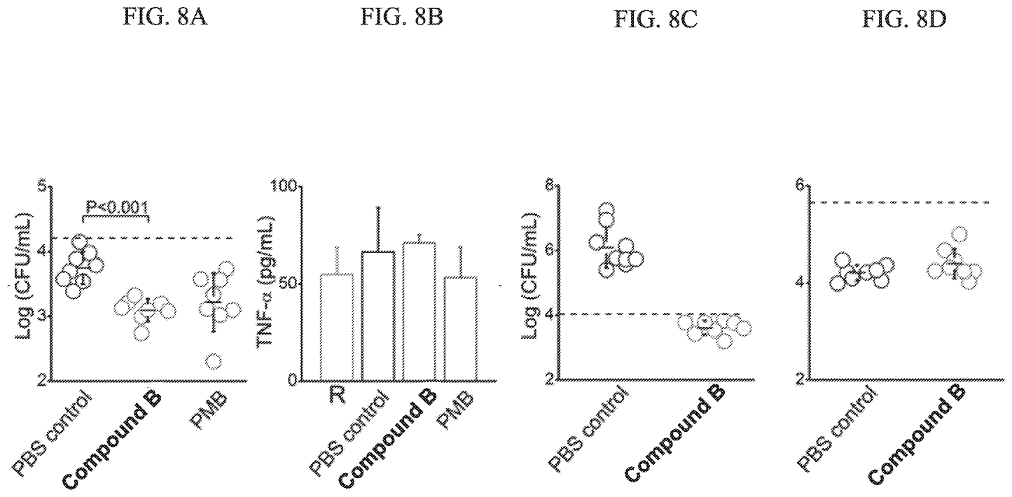

In this respect, however, PMB nonapeptide exhibited antibacterial activity in human but not mouse serum (perhaps because diluted serum was used). Also noteworthy is the fact that supplementation of human serum with lysozyme or lactoferrin resulted in similar outcomes as in mouse serum in terms of the synergy between Compound B and lysozyme for both E. coli (FIG. 4C) and K. pneumoniae (FIG. 5C). However, unlike in culture medium, where Compound B was clearly devoid of antibacterial activity (Table 1 and FIG. 6), Compound B-treated serum exhibited significant growth inhibition in absence of exogenous lysozyme, whereas a potent bactericidal effect was observed in its presence. Thus, given the significant endogenous levels of complement and lysozymes in mammalian sera, the findings presented herein support the possible implication of these compounds (and, conceivably, other serum-soluble antibacterial compounds) in mediating the eventual induction of in vivo antibacterial activity of bioavailable compounds that have the capacity to damage the outer membrane, despite being devoid of antibacterial activity themselves. The results presented in FIG. 7 and FIG. 8 support this view.

FIGS. 6A-B present growth kinetics assessed by measuring the absorbance at 620 nm of E. coli 25922 (FIG. 6A) and K. pneumoniae 1287 (FIG. 6B) in absence or presence of the 10 μM of Compound B (circles denote vehicle control, triangles denote Compound B; error bars represent standard deviations).

FIGS. 7A-B present mouse peritonitis-sepsis model, wherein FIG. 7A shows survival of neutropenic ICR mice (10/group) infected intraperitoneally with Escherichia coli 25922, 1.2×10⁶ CFU/mouse or Klebsiella pneumoniae 1287, (0.78±0.05)×10⁷ CFU/mouse (left and right, respectively) and treated subcutaneously with Compound B, 1 hour or 1 and 6 hours after inoculation, wherein the right panel, data points represent average from 2 independent experiments (standard deviations were less than 10%), and wherein FIG. 7B shows a variant assay where neutropenic ICR mice (10/group) were infected intraperitoneally with untreated (control) or pretreated E. coli 25922, (1.3±0.283)×10⁶ CFU/mouse or K. pneumoniae 1287, (9.75±0.354). 106 CFU/mouse, and in Compound B-treated groups bacteria were pre-incubated in vitro with 5 μM Compound B for 15 minutes (plotted are the surviving mice after 3 days post-infection).

FIGS. 8A-D present the results obtained for the thigh-infection model, wherein normal mice (8/group) were inoculated intramuscularly with Escherichia coli 25922 (panel a), Klebsiella pneumoniae 1287 (FIG. 8C) or MRSA USA300 10017 (FIG. 7D), and treated subcutaneously 1 hour thereafter (dashed lines represent the inoculums; data points represent the CFU counts obtained after homogenizing the thighs of mice euthanized 24 hours post-treatment), and wherein FIG. 8B shows TNF-α blood levels as determined by ELISA 24 h after E. coli infection in treated, untreated and uninfected mice (Compound B at 12.5 mg/kg body weight; R denotes reference plasma from uninfected mice).

Compound-Mediated Protection from Sepsis:

Under the experimental settings previously described, Compound B revealed a potent capacity to counteract the induced disease course. FIG. 5A shows that, after administration of a single dose, Compound B protected 40% of E. coli-infected mice from developing fatal sepsis; infection with this highly virulent pathogen resulted in the death of 100% of vehicle-treated mice. Moreover, multidose experiments revealed that a lower dose administrated twice further increased the survival rate to 70%. A comparable outcome was obtained with another species representing medically relevant gram-negative bacteria, K. pneumoniae, with survival frequencies of 60% and 90% after administration of similar single and double doses, respectively, to mice, thereby reinforcing the postulated efficacy of Compound B monotherapy against gram-negative bacteria and suggesting its potentially improvable effectiveness via optimization of the treatment regimen.

This efficacy level is remarkable since such systemic efficacy against gram-negative bacteria, exerted by a compound devoid of antibiotic activity, has hitherto been unreported. The closest case reports involved the use of a mouse model in which E. coli infection and treatment were both performed intraperitoneally, with significant reduction in growth achieved despite the compound's inefficient antibiotic activity in vitro. However, since peptide administration was performed 4 hours before infection, this implies that the compound fraction interacting with bacteria was excessively low. Consequently, this compound acts by a different mechanism (i.e., by activation of cellular immunity), as suggested herein.

Evidence for direct interaction of Compound B with the test bacteria in vivo was obtained using an analogous experiment, which assessed in vivo efficacy under conditions where the compound-bacteria interaction is unquestioned. First, bacteria were exposed to Compound B (5 μM for 15 minutes) and then inoculated onto neutropenic mice. As shown in FIG. 5B, mortality induced by the pretreated bacteria was significantly prevented (animal survival increased from 10% to 40% and from 35% to 70% for infections by E. coli or K. pneumoniae, respectively).

These findings support a cause-and-effect relationship between direct compound interaction with bacteria and survival of infected mice (FIG. 5A). FIGS. 6A-B show the growth kinetics of strains used in vivo, as monitored in vitro. The practically identical curves obtained in the presence and absence of Compound B (P=0.4 and 0.9, respectively) join the data presented in Table 1 in confirming that the observed in vivo efficacies are unlikely to stem from the direct antibiotic activity of Compound B since its blood concentrations are unlikely to approach growth-inhibitory levels (observable at more than 50 μM in culture medium). These findings therefore indicate that some antibacterial cofactor(s) is required to explain the compound-mediated efficacy. Thus, combined with the data from the previous sections, the findings suggest that, by minimizing nonspecific interactions with multiple amphipathic/anionic tissue constituents, Compound B has achieved the high circulating levels necessary to attain the inoculated pathogens and neutralize their disease inducing capacity.

Also investigated was the possibility that Compound B, that mimics host defense peptides (HDPs), can affect bacterial viability in mice by recruiting cellular immunity component(s), since various HDPs and HDP-like compounds were reported to exert various immunomodulatory activities. For this purpose, the inventors used the thigh infection model, created using nonlethal inoculums for inducing intramuscular infections in normal (non-neutropenic) mice, and assessed the viability of inoculated bacteria after systemic treatment. Compound B and PMB reduced the number of inoculated E. coli by 80% and 64%, respectively (FIG. 8A), under conditions in which the initial inoculum in vehicle-treated control mice was nearly unchanged, reflecting the phagocytes' ability to limit proliferation of the inoculated bacteria. Under the same conditions, Compound B also significantly inhibited proliferation of K. pneumoniae (FIG. 8C).

PMB, a highly toxic “last resort” antibiotic, used herein as a reference antibiotic because of its potent bactericidal activity against gram-negative bacteria, was nearly as efficacious as Compound B, although its in vivo activity might stem from a direct bactericidal mechanism, immune sensitization, or a combined effect. Nonetheless, the fact that a bactericidal antibiotic did not reduce the CFU count more than Compound B (which is devoid of antibiotic activity) raises the possibility that in vivo, PMB is not necessarily bactericidal but might merely facilitate the antibacterial activity of serum components, as proposed for Compound B. Extending the comparison to toxicity issues, noteworthy is the finding that subcutaneous administration of Compound B did not result in any visible adverse effect (e.g., signs of discomfort or stress) at the highest tested dose (i.e., 20 mg/kg body weight), a dose at which PMB was reported to cause zero mortality as well.

Also, under the tested conditions, both Compound B and PMB failed to produce a significant change in the systemic levels of tumor necrosis factor α (a major immune marker orchestrating the host innate responses to infection, as measured prior to and 24 hours after infection with E. coli (FIG. 8B) or Klebsiella, even at 100-fold higher inoculum, thereby arguing against the involvement of activated pro-inflammatory pathways in the observed outcome. The fact that in vivo efficacies of Compound B (FIGS. 7A-D) were observed under neutropenic conditions also argues against a critical role played by the host immune cellular arm, although other cell types might have fulfilled the leukocytes' role. Thus, the involvement of cellular immunity remains unsettled and requires additional studies. Regardless, the fact that Compound B can similarly affect the CFU counts of both E. coli and K. pneumoniae but not S. aureus (FIG. 8D), supports the notion that the effect of Compound B is directed against gram-negative species whose lipopolysaccharide may leach (because of outer membrane damage, as evidenced in FIG. 1A) and stimulate the local recruitment of yet undetermined innate immune factors.

The combined data presented hereinabove provide evidence for the ability of a small linear compound to control gram-negative bacteria infections systemically, while being devoid of antibiotic activity. The molecular basis for this effect is yet ill understood, but the surprising findings presented herein suggest a plausible role for the compounds provided herein as a membrane-active compounds that render bacteria vulnerable to humoral antibacterial factors. The circulating concentration of the compounds provided herein required for this activity (i.e., less than 10 μM) is reasonably attainable at nontoxic doses. Other lipopeptides, such as polymyxins, might achieve a similar biomedical potential, although the design of the compounds provided herein may present advantages, as demonstrated in terms of synergistic efficacy or their molecular simplicity.

Beyond the specific attributes of the compounds provided herein, this study also suggests that, to provide effective protection against gram-negative bacteria in vivo, HDPs are not required to exert bactericidal activity. Both experimental data and logic support this view. Indeed, the canonical mammalian HDPs defensins or cathelicidins often exhibit rather high MICs and/or bactericidal values. These characteristics argue against their touted critical role in direct bactericidal activity, suggesting that they need only to overcome the outer membrane permeability barrier to expedite the action of bactericidal humoral and/or cellular immune components. Such a mechanism may be advantageous as its milder action reduces the risk for complications associated with endotoxins released by bactericidal compounds, thereby promoting a smoother restoration of homeostasis. In this sense, borderline-hydrophobic membrane-active compounds may present an advantage over outright hydrophobic counterparts.

### Example 3

### Eliciting Improved Antibacterial Efficacy

Membrane active compounds (MACs) having the ability to target multiple bacterial functions simultaneously has attracted increasing interest for their potential to overcome infections while avoiding diverse resistance mechanisms. Unlike outright hydrophobic bactericidal MACs, borderline hydrophobic analogs tend to cause a variety of superficial impairments, ranging from barely detectable injuries to full-fledged compromising harms with bacteriostatic consequences. Being associated with rather high minimal inhibitory concentrations (MICs), borderline hydrophobic MACs can be transparent to many antibiotic screens while triggering damages of relatively high metabolic cost by altering membrane attributes such as bilayer thickness, charge, or fluidity. Functional membrane constituents can be chemically amended or sterically distorted to a point that allows proton leakage, which in turn will affect (at least temporarily) the transmembrane chemical potential required for vital bacterial functions such as bioenergetics, transport, and/or communication. A potentially exploitable consequence is that while engaged in repair processes, such bacteria are de facto rendered vulnerable to otherwise inefficient antimicrobials, including efflux substrates and low-permeability antibiotics.

Being a class of synthetic cationic MACs, the compounds presented herein can sensitized gram-negative bacilli (GNB) to various antibiotics in correlation with mild membrane damage. In the studies presented hereinabove, Compound B with enhanced bioavailability is shown capable of sensitizing GNB to host plasma immune factors. The study presented below investigates whether the bioavailability of Compound B can further improve the therapeutic outcome in combination with ineffective antibiotics. In other words, it is verified that the compounds presented herein indeed disturb the outer membrane (OM) functions, and whether this trait can be translate into the capacity to potentiate antibiotics suffering from access or efflux issues. In such case, in vivo treatments might simultaneously benefit from both endogenous and exogenous antibacterial systems.

Hence, the study below present in vitro and in vivo evidence supporting the notion that the compounds presented herein, and exemplified by Compound B, enhances the antibiotic performance of medically relevant representatives of such antibiotics, exemplified by rifampin and erythromycin.

Evidence for Membrane Damage:

The compounds' aptitude to alter the OM structure at sub-inhibitory concentrations was revisited herein by using various complementing methodologies. First it was attempted to visualize the alleged membrane damage resulting from E. coli exposure to Compound B in PBS at concentrations likely to be achieved in mouse blood at nontoxic doses (i.e., at least 10 mM) by using electron microscopy to compare the contour of untreated and treated bacteria. However, clear topological damages failed to materialize despite attempts to obtain high resolution SEM images (data not shown). It is possible that the resolution may be not high enough to see the difference; the aggressive sample preparation steps required by this technique might contribute to minimizing the surface differences eventually occurring between treated and untreated bacteria.

In contrast, two distinct biochemical assays support the notion that Compound B significantly altered the E. coli outermost permeability barrier. As shown hereinabove, E. coli mutant strain ML-35p was used to monitor the selective permeation of GNB outer and/or cytoplasmic membrane (CM). This study provided initial evidence for the compound's ability to damage both membranes, although asymmetrically (i.e., some CM permeabilization occurred only at 10 mM). To confirm the occurrence of such damage specifically in the ATCC strain 25922 used in animal studies, the OM-impermeable hydrophobic fluorescent dye NPN was used; this agent is able to bind the CM only upon OM disruption, thereby enhancing the fluorescence emission intensity.

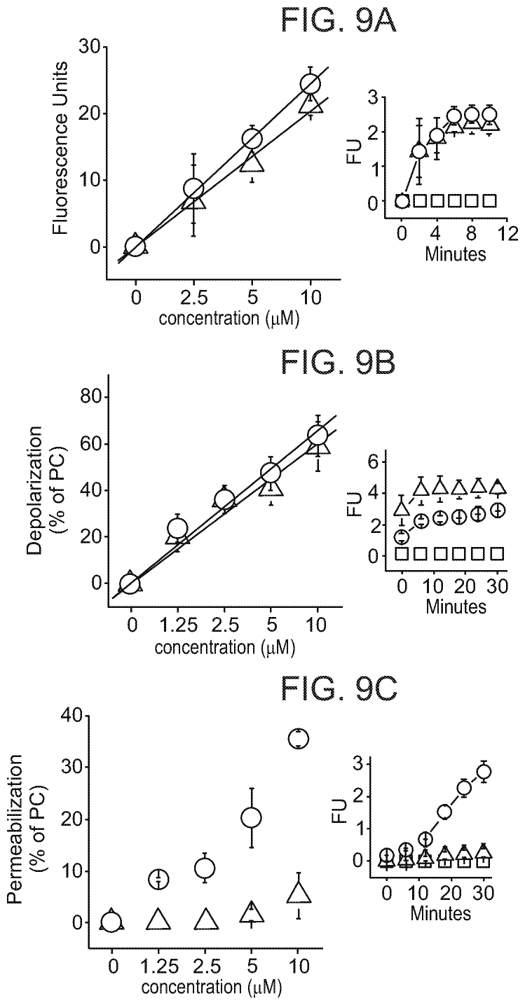

FIGS. 9A-C show evidence for membrane damages to E. coli 25922, wherein FIG. 9A presents time- and dose-dependent data supporting OM permeabilization as evaluated 6 minutes after exposing bacteria to Compound B or PMB in the presence of hydrophobic fluorescent dye NPN, FIG. 9B presents similar data supporting CM depolarization upon pre-incubation of bacteria with potential-sensitive dye (DiSC₃5) and ulteriorly treated with Compound B or PMB, and FIG. 9C presents CM permeabilization data obtained using DNA binder (ethidium bromide) in the presence of Compound B or PMB (data points taken at t=20 minutes; insets show representative kinetics, using 0 and 10 mM Compound B or PMB; positive control (PC) for full depolarization and permeabilization was achieved with C12K7α8 (50 mM) [Rotem, S. et al. FASEB J., 2008, 22, 2652-2661] (FU denotes fluorescence units; triangles denote Compound B, circles denote PMB, squares denote untreated control; error bars=SD).

As can be seen in FIG. 9A, sub-MIC concentrations of Compound B rapidly caused a dose-dependent increase in fluorescence, similar to that caused by polymyxin B (PMB), a reference-standard potent antibiotic, whose high-affinity interaction with LPS triggers a bactericidal mechanism against GNB. The results also confirmed a previously suspected result, pertaining to a possible mild permeabilization of ML-35p inner membrane. Thus, using the membrane potential sensitive dye DiSC₃5, it is shown in FIG. 9B that at low Compound B concentrations (more than 1 mM), the fluorescent signal emitted by treated bacteria has increased in a rapid and dose-dependent manner, suggesting that Compound B has caused a partial depolarization of the CM (seemingly as did PMB). In contrast, the bactericidal PMB has also increased significantly more than Compound B, with the CM permeability to molecules larger than protons, as demonstrated with ethidium bromide (FIG. 9C).

Collectively, these findings ratify the view that Compound B breaches the E. coli permeability barrier functions of both OM and CM at low micromolar concentrations. Whereas the OM damage may not involve major disturbances of the outermost LPS layer (at least not observable by SEM), the damage was significant enough to increase OM permeability to hydrophobic small molecules such as nitrocefin (in ML-35p) and NPN (in ATCC strain 25922). In contrast, the damage sustained by the CM conforms to the notion of a superficial, repairable injury. Interestingly, because the positively charged compound seems able to depolarize the CM (FIG. 9B), this could hint that Compound B might go through the OM in a passive manner (without necessarily forcing its way through by physically damaging it). Indeed, this would fit well with the SEM data. However, data shown in FIG. 9A, as well as results from the above-presented studies, argue against such a possibility. In fact, even less hydrophobic analogs (e.g., C₈KKC₁₂K) were able to damage the OM.

In Vitro Evidence for E. coli Sensitization to Antibiotics:

The current study was extended under the assumption that the compounds presented herein did increase the OM permeability, thereby inducing the repair mechanism. Because during the repair process these bacteria become sensitive to diverse antibacterial compounds, the example presented below set out to evaluate bacterial sensitization to ineffective antibiotics by determining the antibiotic potency changes induced by Compound B.

To define the compounds' capacity to increase bacterial permeability to typical antibiotics, first investigated was the RNA polymerase B inhibitor, rifampin, which is normally used for treating Mycobacterium infections. It is, however, often ineffective on enteric bacteria as a result of its poor capacity to cross their OM. To exploit this weakness, checkerboard-type experiments were performed, exposing E. coli to rifampin's increasing concentrations in the presence of constant levels of Compound B, which would indicate that an eventual reduction in the MIC value would testify to the compound's capacity to increase rifampin's permeability across the OM.

Table 2 presents data showing the synergistic effect of Compound B with antibiotics. Shown in parentheses are calculated sensitization factor, defined as antibiotic's MICs ratio (in absence vs. presence of Compound B) at specified Compound B concentration. MICs of Compound B against 5 listed strains were invariably higher than 50 mM.

TABLE 2

| | | Antibiotic MIC (μg/ml) in the presence of Compound B (μM) | | | | | | | | |
|---|---|---|---|---|---|---|---|---|---|---|
| Antibiotic | E. coli strain | 0 | 1.25 | | 2.5 | | 5 | | 10 | |
| Rifampin | 25922 | 8-16 | 1 | (8-16) | 0.063 | (127-254) | 0.004 | (2000-4000) | 0.001 | (8000-16,000) |
| | 35218 | 8 | 2 | (4) | 0.125 | (64) | 0.016 | (500) | 0.002 | (4000) |
| | 14182 | 8 | 2 | (4) | 0.031 | (258) | 0.004 | (2000) | 0.002 | (4000) |
| | 16327 | 16 | 2 | (8) | 0.031 | (516) | 0.004 | (4000) | 0.002 | (8000) |
| | 14384 | 8 | 0.125 | (64) | 0.016 | (500) | 0.008 | (1000) | 0.002 | (4000) |
| Erythromycin | 25922 | 128 | 8 | (16) | 2 | (64) | 0.5-1 | (128-256) | 0.25 | (512) |
| | 35218 | 128 | 64 | (2) | 4 | (32) | 1 | (128) | 0.5 | (256) |
| | 14182 | 128 | 128 | (1) | 16 | (8) | 4 | (32) | 1 | (128) |
| | 16327 | 512 | 64 | (8) | 4 | (128) | 1 | (512) | 0.5 | (1024) |
| | 14384 | >512 | 64 | (>8) | 2 | (>256) | 1 | (>512) | 0.5 | (>1024) |

As can be seen in Table 2, Compound B manifested high capacities for sensitizing bacteria, as evidenced by high sensitization factors (defined as the antibiotic's MIC ratio in the presence of a specified agent concentration versus the MIC obtained in its absence). For example, in the presence of 10 mM Compound B, the sensitization factor of E. coli strain 25922 was 16,000 because rifampin's MIC value was reduced from 16 μg/ml to 1 ng/ml. Essentially similar results were obtained with 4 additional strains where the sensitization factors increased by up to 4000- or 8000-fold (see, Table 2).

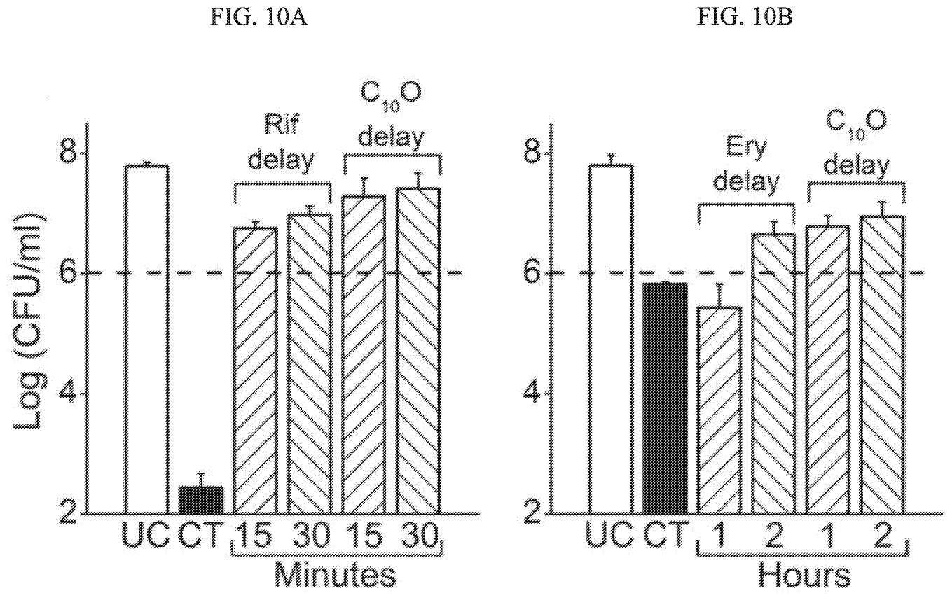

Next investigated was the case of erythromycin, a macrolide antibiotic whose interaction with the 50S ribosomal subunit inhibits protein synthesis. Although erythromycin can easily cross the OM through the porin system, it is less effective on GNB because its cytoplasmic accumulation is prevented by the resistance—nodulation—division efflux pump. Again, the test strategy exploited this fact, it has been predicted that erythromycin's antibiotic activity would increase if the compound-induced depolarization (FIG. 9B) would reduce the E. coli efflux rates by limiting its proton based energy source. The results supports this notion: the lower part of Table 2 shows that in the presence of Compound B, erythromycin's potency over all strains tested was highly enhanced (less than 2, and even more than 3 orders of magnitude), albeit generally less than observed for rifampin (i.e., whose sensitization factors increased by 3 or 4 orders of magnitude). This difference probably reflects the antibiotics' mechanistic differences, namely the low copy numbers of RNA-polymerase that rifampin is required to shut down in order to achieve bacterial death. Also noteworthy is the fact that all the strains tested were significantly more resistant to erythromycin (i.e., MICs of more than 128 μg/ml, as opposed to 8-16 μg/ml rifampin in the absence of the compound provided herein). If this resistance level is caused by drug efflux (particularly because the OM does not represent a major permeability barrier for erythromycin), then the compound's ability to induce CM depolarization (FIG. 9B) could support a sensitization mechanism based on limiting the efflux function. In that case, the drug's potency would emerge from the newly achieved ability to linger on in the cytoplasm, long enough to inhibit its ribosomal target. The fact that Compound B exhibited significantly higher antibacterial activity on a resistance—nodulation—division deletion mutant compared to its wild-type isogenic strain (i.e., MIC=6.3 and more than 50 μM, respectively; data not shown) provides a strong argument in support of this view. This effect also seems to subsist on assessing antibacterial activities of the synergistic pairs (i.e., Compound B+rifampin and Compound B+erythromycin) by comparing the number of CFUs in cultures exposed to the compound+antibiotic simultaneously, as opposed to delaying the addition of one or the other, as summarized in FIG. 10.

FIGS. 10A-B present results of simultaneous versus delayed drug exposure assays, wherein E. coli 25922 was exposed in fresh LB culture medium to both Compound B (10 mM) and antibiotic without delay (CT) or after delaying exposure for specified time periods to 0.06 μg/ml rifampin (FIG. 10A) or 4 μg/ml erythromycin (FIG. 10B), whereas CFU counts were determined after additional 3 hours incubation in LB (UC denotes untreated control, CT denotes combined treatment, Rif denotes rifampin; C₁₀O denotes Compound B, Ery denotes erythromycin; dashed line represents inoculum; error bars=SD).

In the case of rifampin (FIG. 10A), at 0.06 μg/ml, the drug reduced the CFU count nearly by 4 log units only in the presence of Compound B (10 μM). In contrast, any delay, even by 15 minutes (of compound or antibiotic), nearly abolished the sensitization effect, demonstrating that optimal synergism requires the simultaneous presence of both compounds, possibly as a result of some rapidly resolved OM damage that facilitates rifampin's permeability.

Interestingly, this was not the case of erythromycin (FIG. 10B), because its delay by up to 60 (but not 120) minutes revealed maintenance of the sensitization activity, possibly reflecting a longer time required to accomplish sufficient repair of the depolarized CM. Also, the fact that Compound B and the macdrolide antibiotic were not mutually reciprocal (unlike the case of rifampin, because delaying Compound B, even by 15 minutes, resulted in the loss of most of the antibacterial activity) provides additional support to this hypothesis.

Although this intriguing issue remains unsettled, the current data indicate that these two a priori weak antibiotic agents have benefited from the compound's membrane-active properties and have become significantly more potent as a result of their capacity to overcome the E. coli natural resistance mechanisms for limiting permeability of the hydrophobic rifampin and we next asked whether these benefits would resist challenges imposed by the complex plasma medium.

Induced Synergism in Plasma:

To address this issue, the capacity of the compounds provided herein to elicit a bactericidal activity was compared in broth and plasma by determining bacterial survival in the presence of the exemplary Compound B (0.6 or 10 μM), rifampin (1 μg/ml), erythromycin (3 μg/ml), or some combination. Results are shown in Supplemental FIG. 1; main findings are summarized in FIG. 4.

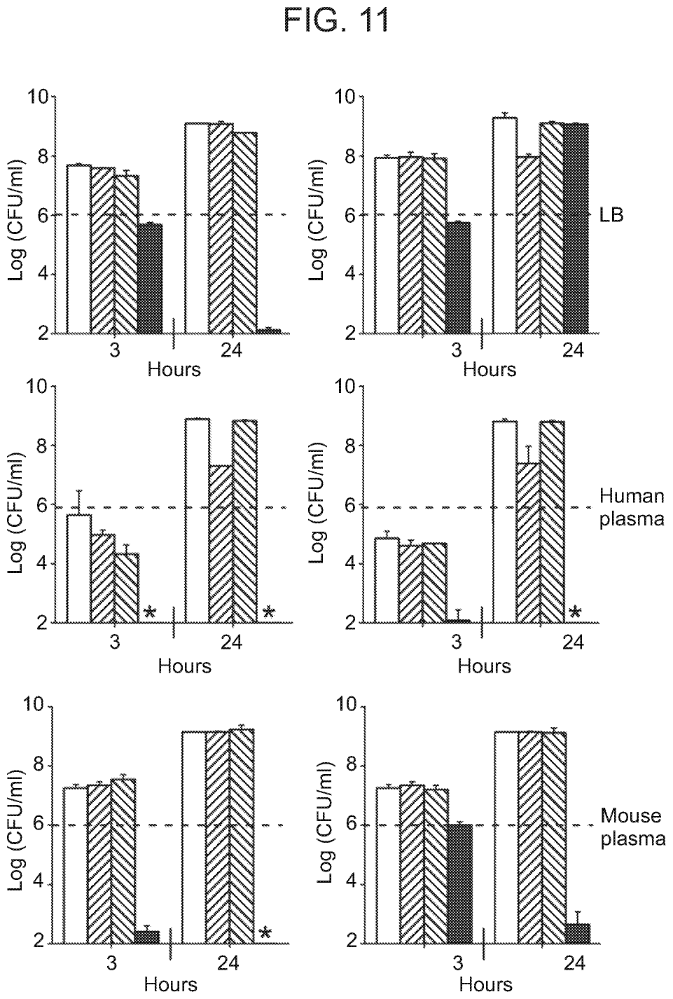

FIG. 11 presents results of a bactericidal kinetic assays conducted in broth versus plasma, wherein the left panels depict time-kill experiments using E. coli 25922 exposed for the specified time periods to Compound B (C₁₀OOC₁₂O; right strips) and rifampin (left strips) or their combination (Grey), and wherein the right panels depict the same experiment where erythromycin substitutes for rifampin (vehicle-treated controls are represented in white columns; dashed line represents the inoculum; asterisk indicates values below detection limit; concentrations: Compound B, 0.6 μM in LB and Human plasma, 10 μM in mouse plasma; Rifampin, 1 μg/ml; Erythromycin, 3 μg/ml; error bars=SD).

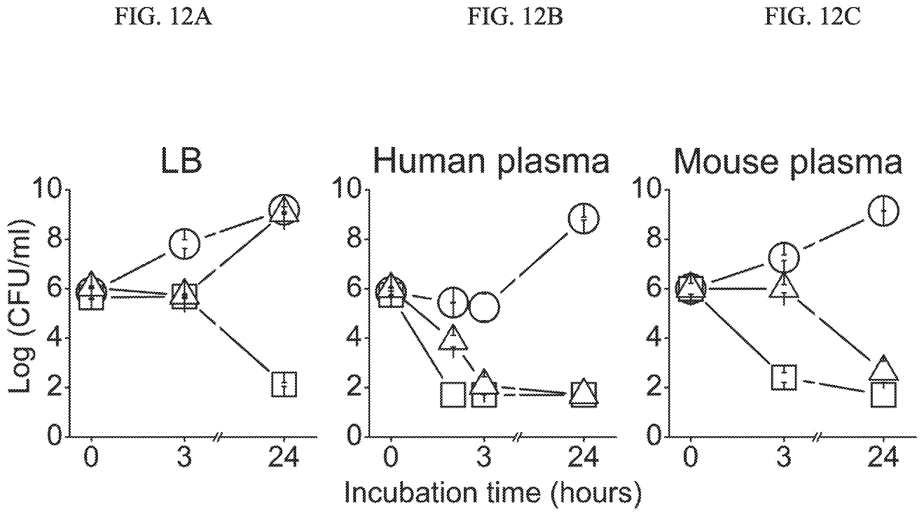

FIGS. 12A-C present broth vs. plasma bactericidal kinetics, wherein time-kill studies of E. coli 25922 exposed to vehicle only (denoted by circles), combination of Compound B plus rifampin (denoted by squares), or Compound B plus erythromycin (denoted by triangles) (concentrations: Compound B, 0.6 μM in LB broth and human plasma, 10 μM in mouse plasma; rifampin, 1 μg/ml; erythromycin, 3 μg/ml; error bars=SD).

In the simpler (broth) medium, none of the individual compound was more active than the vehicle-treated control (FIG. 11), whereas the combined treatments initially (after 3 hours exposure) fully inhibited bacterial growth but then diverged in their ability to affect viability at the 24-hours end point. Thus, only the presence of rifampin ultimately reduced bacterial survival by more than 99%, unlike erythromycin, which did not alter the CFU count.

Performing this experiment in human plasma, however, drastically transformed these outcomes. Thus, while the plasma itself has transiently limited bacterial growth, the individual compounds were not particularly more active than the vehicle-treated control (FIG. 11). However, both combined treatments exhibited potent bactericidal activities, achieving nearly 100% death of inoculated bacteria. These experiments highlighted two interesting observations: firstly, because on its own Compound B is unable to reduce bacterial survival, these findings demonstrated the persisting MAC activity in plasma, reflected in the capacity to induce bactericidal activities as observed in broth; and secondly, the data indicated that the higher potency observed in plasma (compared to broth) emanated from the host's antibacterial factors, which under the experimental conditions managed only to limit bacterial proliferation for several hours and were potentiated in the presence of the combined treatments. Thus, in the presence of rifampin, the CFU count was reduced by more than 3 log units within 2 hours of exposure (as opposed to about null in broth), whereas in the presence of the bacteriostatic erythromycin, the bactericidal rate was practically similar to rifampin, albeit accomplishing the feat within 3 hours instead of 2 hours. These findings therefore enforce the notion that through the OM damages instigated by the compounds provided herein, the plasma-resistant E. coli were rendered more sensitive to both the endogenous antibacterial proteins and by the exogenous antibiotics (as represented by rifampin and erythromycin).

Remarkably, the outcomes express synergism rather than additive effects of the individual compounds. Repeating these experiments in mouse plasma supported this view. Because antibacterial activity of mouse plasma is not as potent as that of human plasma, as previously observed, a higher compound concentration was used (i.e., 10 instead of 0.6 μM) to compensate for that, and still show evidence of synergism. The results demonstrated that the mouse plasma alone was unable to significantly limit bacterial proliferation (FIGS. 12A-C), whereas treatments combining Compound B with rifampin or erythromycin have in both cases expressed synergism of action when evaluated at the 24 hours end point, where they reduced bacterial survival significantly more than in broth.

Enhancing In Vivo Efficacies Through Combination Therapies:

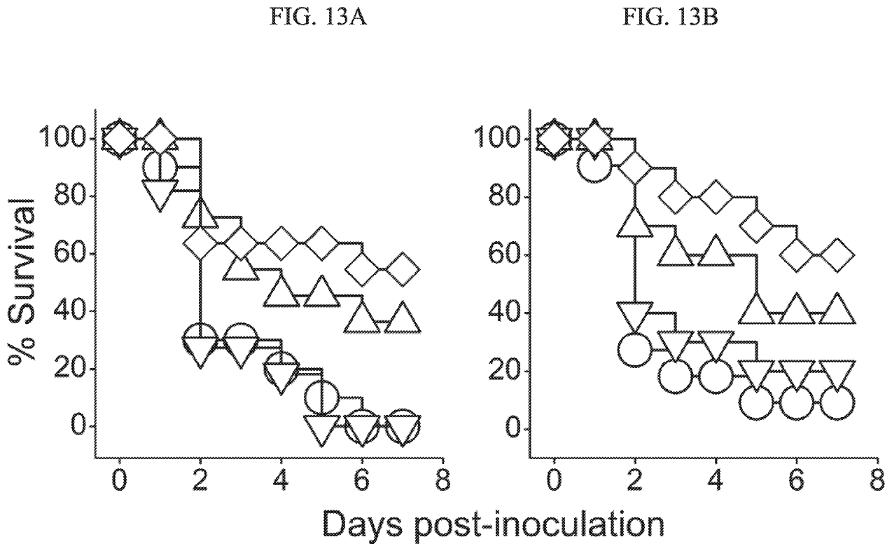

The above outcome prompted the extension of the investigation by testing the hypothesis in vivo, using a mouse model of infection, and the results are presented in FIGS. 13A-B).

FIGS. 13A-B present the results of single versus combination therapy using mouse peritonitis-sepsis model, showing survival kinetics of neutropenic ICR mice (n=10 mice/group) infected intraperitoneally with E. coli 25922 (1.3±0.2×10⁶ CFU/mouse), wherein one hour after infection, mice were treated s.c. with Compound B and/or rifampin (FIG. 13A) or with Compound B and/or erythromycin (FIG. 13B), whereas rifampin was administered orally immediately after inoculation (circles denote vehicle control, inverted triangles denote 20 mg/kg rifampin or 100 mg/kg erythromycin, triangles denote 12.5 mg/kg Compound B, diamonds denote combination of Compound B+rifampin or Compound B+erythromycin).

To assess bacterial sensitization under in vivo conditions, mouse peritonitis-sepsis model was used, where neutropenic mice were infected with E. coli, applying an inoculum size previously determined to induce death within 24 to 48 hours if untreated. The cytoplasm-targeting antibiotics rifampin or erythromycin (seldom prescribed against GNB) were used to test the compound's ability to overcome the permeability barriers of the OM and/or CM, respectively, under in vivo conditions, as determined by comparing efficacy of single versus combination therapy. To select for antibiotics dose, schedule, and administration route, taken into account were published data aiming to synchronize their maximal circulating concentrations by weighing their respective pharmacokinetic profiles as follows: the compound's blood concentration time course is bell shaped (see, Table 3), gradually increasing for rifampin and gradually decreasing for erythromycin. Therefore, to compensate for this pharmacokinetic heterogeneity, rifampin was administrated orally immediately after infection, followed by subcutaneous administration of Compound B 1 hour after inoculation, so as to allow their active concentrations (as determined in vitro) to favorably coincide. In contrast, because of its rapidly decreasing blood levels, erythromycin was administered at the same time as Compound B (i.e., 1 hour after infection) for maximal drug exposure time.

TABLE 3

| | Route of | Dose | Plasma concentration (μg/ml) | | |
|---|---|---|---|---|---|
| Test agent | Administration | (mg/kg) | 0.5 h | 1 h | 2 h |
| Compound B | Subcutaneous | 12.5 | 5.2 ± 0.7 | 11.1 ± 0.8 | 6.2 ± 0.5 |
| Erythromycin | Subcutaneous | 25 | 3.7 | 2.3 | 0.4 |
| Rifampin | Oral | 20 | 6.5 ± 4.7 | 10.3 ± 0.4 | 13.2 ± 4.4 |

FIG. 13A shows that rifampin administration (20 mg/kg) resulted in a zero survival rate of infected mice (similar to vehicle-treated control), whereas its combination with Compound B increased mice survival significantly more than observed with Compound B alone. Thus, on its own, Compound B (dosed at 12.5 mg/kg) was able to protect 36% of E. coli-infected mice from developing sepsis compared to 55% protection observed upon combination (P, 0.01).

Equivalent data obtained in another experiment assessing the effect of combining Compound B and erythromycin again demonstrated that the drugs were clearly more effective in combination compared to their individual administrations (FIG. 13B). Thus, the in vivo data seem to join the broth and plasma data sets in supporting the notion that the observed enhanced efficacies resulted from simultaneous contributions of the antibiotics and the host immune system, where Compound B allegedly plays a facilitating/eliciting role in both cases.

In summary, while Compound B is proposed to sensitize GNB to host plasma antibacterial proteins, this study provides evidence for the compound's capacity to perform much better in the presence of an antibiotic, bestowing potent antibacterial activity onto virtually inactive endogenous and/or exogenous factors and agents. In this sense, the data presented herein ratifies the conclusions drawn in the studies presented hereinabove and elsewhere, that propose that the newly found antibacterial potencies are likely to stem from the damaged OM and/or CM, thereby supporting this approach's potential usefulness in the search of new alternatives to antibiotics.

### Example 4

### Toxicity

The compounds presented herein are compared against PMB in terms of time- and dose-dependent effects in cultures of mouse and human cells, as well as in normal mice. Various cell types, including HaCaT keratinocytes, Hsf fibroblasts and blood cells (neutrophils and macrophages) are cultured in presence of twofold dilutions of the tested compound (generally, 50 μM to zero) and their metabolic activity/viability evaluated using MTT assay. Hemolysis is similarly assessed by determining hemoglobin release of washed human and mouse erythrocytes. In addition, the drugs intracellular uptake by normal kidney cells will be compared with Megalin receptor knockout cells where the drugs identity and quantity are determined by quantitative LC-MS analysis after cells lysis, filtration and extraction. LC-MS is used also to determine the blood and urine drug concentrations following administration to normal mice. The resulting data are verified against acute toxicity studies in normal mice that will determine their MTD for several routes of administration (IV, IP, SC). Plasma samples obtained before- and after-inoculation/treatment are submitted to a comprehensive robot analysis and of toxicity biomarkers such as KIM-1 and α-GST.

Systemic efficacy of the compounds presented herein, that combine high antibacterial activity in plasma and low toxicity are assessed for the capacity to resolve infections systemically, using at least two mouse-infection models routinely employed in the lab (the peritonitis-sepsis model, which determines animal survival upon infection with lethal inoculums, and the thigh-infection model, which assesses the treatment's ability to reduce bacterial load in mice muscles infected with non-lethal inoculums), including different routes of administration, dose regimens and comparing normal versus neutropenic mice. If deemed appropriate, two additional models are tested (lung-infection and bacteremia). To overcome the fact that mouse plasma displays weak antibacterial potency (compared to human plasma) the plasma potency is artificially enhance by testing in vivo efficacy in mouse infection models using combination therapy strategies, as in previous studies, where the antibiotics will substitute for the role of innate antibacterial proteins. These in vivo tests are supplemented with in vitro treatments of infected human blood and plasma.

The compounds, according to the present invention, have been found non-toxic.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference.

In addition, citation or identification of any reference in this application shall not be construed as an admission that such reference is available as prior art to the present invention. To the extent that section headings are used, they should not be construed as necessarily limiting.

1. A compound selected from the group consisting of:

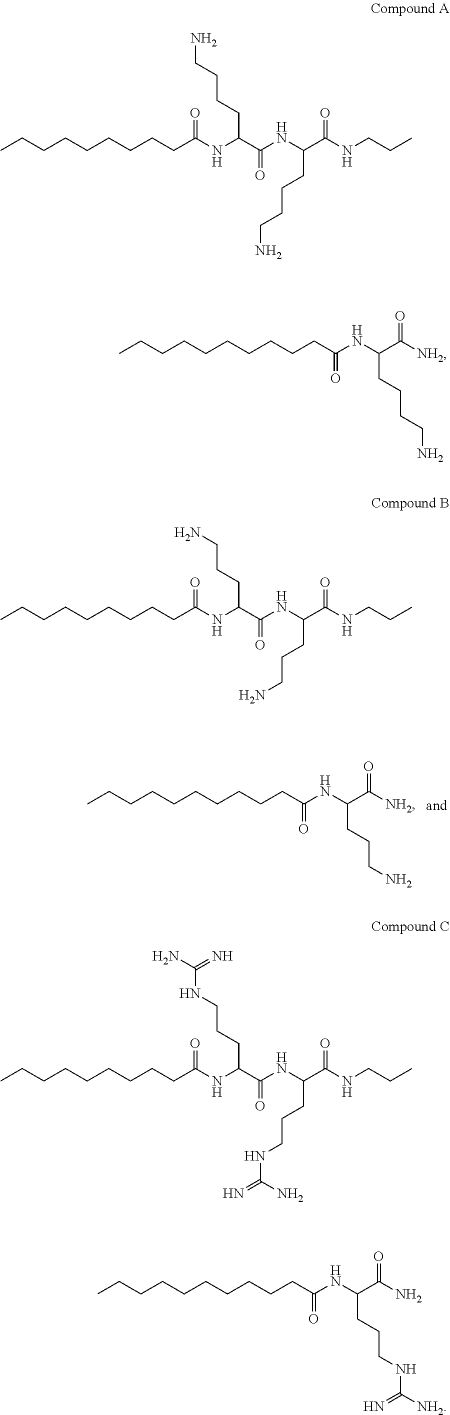

2. The compound of claim 1, being Compound B.

3. A pharmaceutical composition comprising, as an active ingredient, the compound of claim 1, or any enantiomer, prodrug, solvate, hydrate and/or pharmaceutically acceptable salt thereof, and a pharmaceutically acceptable carrier.

4. The composition of claim 3, being packaged in a packaging material and identified in print, in or on said packaging material, for use in the treatment of a medical condition associated with a pathogenic microorganism in a subject.

5. The composition of claim 4, devoid of an antimicrobial agent.

6. The composition of claim 5, further comprising an antimicrobial agent.

7. The composition of claim 6, wherein said antimicrobial agent is ampicillin and said pathogenic microorganism is Yersinia pseudotuberculosis.

8. A method of treating a medical condition associated with a pathogenic microorganism in a subject, the method comprising administering to the subject a therapeutically effective of the compound of claim 1.

9. The method of claim 8, devoid of administering an antimicrobial agent to the subject, wherein said therapeutically effective of the compound is an immunopotentiating amount.

10. The method of claim 8, further comprising co-administering to the subject a therapeutically effective amount of an antimicrobial agent, wherein:
said therapeutically effective amount of said antimicrobial agent is lower than a therapeutically effective amount of said antimicrobial agent when administered without the compound, and
said therapeutically effective amount of compound is a potentiating amount thereof with respect to said antimicrobial agent.

11. The method of claim 10, wherein said antimicrobial agent is ampicillin and said pathogenic microorganism is Yersinia pseudotuberculosis.

12-15. (canceled)
